(19) United States
(12) Patent Application Publication
(10) Pub. No.: US 2005/0114915 A1
(43) Pub. Date: May 26, 2005

(54) Method for sex biasing spermatozoa

(75) Inventors: Barb Ariel Cohen, Watertown, MA (US); Michael F. Morris, Ashland, MA (US)

Correspondence Address: EDWARDS & ANGELL, LLP, P.O. BOX 55874, BOSTON, MA, 02205, US

(73) Assignee: Vicam L.P.

(21) Appl. No.: 10/723,268

(22) Filed: Nov. 26, 2003

(51) Int. Cl. A01K 67/027

(52) U.S. Cl. 800/21

(57) ABSTRACT

A method is described for treating a specimen of semen containing sperm cells to increase the relative number of sperm cells of a desired sex type in a treated specimen to increase the potential for conceiving an offspring of the desired sex. A specimen of semen is treated after a predetermined time to increase the relative ability of a at least a portion of the semen to conceive an offspring of the desired sex. The treatment preferably comprises contacting the sperm cells with an agent that preferentially effects sperm cells of a selected sex type. In some preferred embodiments, the semen is separated into two components. A first component has a higher number of sperm of the desired sex type than sperm of a non desired sex type and a second component has a higher number of sperm of the non desired sex type relative to sperm of the desired sex type. In one embodiment, the separating step is performed after a predetermined percent of the sperm cells exhibit a punctate pattern, which is capable of determination by labeling the sperm cells with Koo antibody and determining the percent of cells labeled. In another embodiment, the separating step is performed after waiting for a time determined by locating a maximum in the curve obtained by plotting percent female cells determined by FISH against percent Koo positive cells, determining the time at which the maximum percent female cells occurs, and beginning the following step no earlier than about one hour before the time of the maximum percent female cells. In yet another embodiment, the separating step is performed after waiting for a period of time between about 2 hours and about 24 hours after collection of the ejaculate. Preferably, in the separating step, the sperm is contacted with a cell binding agent, permitting the sperm of the non desired sex type to preferentially bind to the cell binding agent, and the cell binding agent with preferentially bound sperm of the non desired sex type is separated from non bound sperm.

### FIELD OF THE INVENTION

This invention relates to methods for enhancing the probability of obtaining offspring of a selected sex. More particularly, this invention relates to methods for separation of spermatozoa bearing DNA determinative of one sex from spermatozoa bearing DNA determinative of the other sex by treating the spermatozoa during a window of time when separation of sperm based on sex is preferentially selected.

### BACKGROUND OF THE INVENTION

Farmers and other animal husbandry persons have long recognized the desirability of enhancing the probability of obtaining offspring of a selected sex. In mammals, the male gamete or spermatozoan controls the sex of offspring. Each spermatozoan contains either an X-type or a Y-type sex-determining chromosome. An X-chromosome spermatozoan creates female offspring after fertilization with an oocyte, while a Y-chromosome spermatozoan creates male offspring after fertilization. Methods have been proposed for increasing the percentage of X-chromosome bearing sperm cells or Y-chromosome bearing sperm cells to thereby achieve a greater chance of achieving female or male offspring, respectively.

Previous methods have included, for example, methods based upon density sedimentation (see, for example, Brandriff, B. F. et al. “Sex Chromosome Ratios Determined by Karyotypic Analysis in Albumin-Isolated Human Sperm,” Fertil. Steril., 46, pp. 678-685 (1986)). U.S. Pat. No. 3,687,806 to Van Den Bovenkamp discloses an immunological method for controlling the sex of mammalian offspring by use of antibodies which react with either X-bearing sperm or Y-bearing sperm and utilizing an agglutination step to separate bound antibodies from unaffected antibodies.

U.S. Pat. No. 4,083,957 to Lang discloses a method for alteration of the sex ratio in animal (including human) offspring by separation of the population of spermatozoa into fractions which are different by reason of the sex-linked electrical charge resident thereon. The separation is carried out by bringing the spermatozoa into close association with an electrostatic charge-bearing material having a charge the sign of which is opposite to the sign of a chosen portion of the spermatozoa, that portion which carries the sex determining character of the unwanted sex, so as to attract and thereby to permit that portion to be isolated, or put to a disadvantage in the fertilization of ova. Concern is expressed with the selection of the charge-bearing material, the adjustment of the pH and particle size thereof, and the control of the surrounding medium in relation to its influence on the charge characteristics of both the charge-bearing material and the spermatozoa. Lang teaches that spermatozoa having respectively male or female sex bearing genetic material also have differing electrostatic charges, normally negative for male and positive for female, and uses this teaching for separation of the male and female spermatozoa with charge bearing materials.

U.S. Pat. No. 4,191,749 to Bryant discloses a method for increasing the percentage of mammalian offspring of either sex by use of a male-specific antibody coupled to a solid-phase immunoabsorbant material to selectively bind male-determining spermatozoa, while the female-determining spermatozoa remain unbound in a supernatant.

U.S. Pat. No. 5,021,244 to Spaulding discloses a method for sorting living cells based upon DNA content, particularly sperm populations to produce subpopulations enriched in X- or Y-sperm by means of sex-associated membrane proteins and antibodies specific for such proteins.

U.S. Pat. No. 5,514,537 to Chandler discloses a method and apparatus for the mechanical sorting of mammalian spermatozoa by sex-type, into a fraction enriched in X-chromosome-bearing spermatozoa, and a fraction enriched in Y-chromosome-bearing spermatozoa. Because of their different DNA content, Y-chromosome spermatozoa are on average slightly smaller than X-chromosome spermatozoa. A column is packed with two sizes of beads. The size of the smaller beads is chosen such that, on average, Y-chromosome spermatozoa will readily fit into the interstices between the smaller beads, while X-chromosome spermatozoa, on average, will not readily fit into those interstices. The size of the larger beads is chosen such that the smaller beads will not readily fit into the interstices between the larger beads. A liquid sample containing the sperm is run through a column so that the liquid first encounters the larger beads, and then encounters the smaller beads. The beads act as a sieve, creating a fraction in the larger beads enriched in X-chromosome spermatozoa, and a fraction in the smaller beads enriched in Y-chromosome spermatozoa.

However, these prior art methods often result in insufficient separation of X- and Y-sperm and often damage the sperm, thereby reducing its motility and fertility success rate.

In the commonly owned and assigned U.S. Pat. Nos. 6,153,373 and 6,489,092, improved methods for sex determination of mammalian offspring are provided using antibodies coupled to magnetic particles for separation of spermatozoa. These methods use magnetic separation to provide gentle separation of populations of spermatozoa.

Therefore, there is a need for new methods for separating X-chromosome bearing sperm from Y-chromosome bearing sperm or enriching a specimen of sperm in X-chromosome bearing sperm or Y-chromosome bearing sperm to obtain both good fertility and a good sex bias towards female or male offspring.

### SUMMARY OF THE INVENTION

The invention provides a method for treating a specimen of semen to increase the relative number of a desired sperm sex type in the treated specimen to increase the potential for conceiving an offspring of the desired sex. Thus, in accord with the present invention, a specimen of semen is treated after a predetermined time to increase the relative ability of a at least a portion of the semen to conceive an offspring of the desired sex. The treatment preferably comprises contacting the sperm cells with an agent that preferentially effects sperm cells of a selected sex type.

In certain preferred embodiments of the invention, the treatment of a specimen of semen includes fractionating the specimen into a first component having a higher number of X-chromosome bearing sperm relative to Y-chromosome bearing sperm and a second component having a higher number of Y-chromosome bearing sperm relative to X-chromosome bearing sperm. Depending on the particular application, either component can provide the increase in the relative number of a desired sperm sex type.

We have discovered that there is a period of time, i.e., a sexing window, during which Y-chromosome bearing sperm develop an ability to adhere or bind to cell binding agents in greater proportion than X-chromosome bearing sperm. If the spermatozoa are treated with a cell binding agent in this window, Y-chromosome bearing sperm will adhere or bind preferentially to a cell binding agent whereas X-chromosome bearing sperm will remain preferentially in the fluid. Thus, separating the cell binding agent with preferentially bound Y-chromosome bearing sperm will remove Y-chromosome bearing sperm preferentially leaving a higher percentage of X-chromosome bearing sperm, thereby biasing the remaining non bound sperm for producing female offspring when introduced into a suitable fertile mammal.

In certain embodiments of the invention, the window can be defined based on the appearance of “sticky patches” on the sperm. It has been found that sex biasing can be correlated to the appearance of the “sticky patches” on sperm cells.

The window opens when a sufficient number of sperm cells exhibit the sticky patches so that separation preferentially removes sufficient Y-chromosome bearing sperm so that the remaining non bound sperm is biased to a desired level with X-chromosome bearing sperm. The window closes when a sufficient number of sperm cells exhibit the sticky patches so that separation can no longer provide the desired biased level.

In certain preferred embodiments of the invention, the window opens with the appearance of the sticky patches on at least about 20% of the sperm cells in the semen, preferably at least about 25%, and more preferably at least about 30%, as determined by labeling the sperm with Koo antibody. The window closes when more than more than 40% of the sperm cells have sticky patches, preferably more than 35%, as determined by labeling the sperm with Koo antibody. Treatment to separate the sperm cells in this window of time provides a first component remaining non bound in the fluid having a higher number of X-chromosome bearing sperm relative to Y-chromosome bearing sperm and a second component bound to cell binding agent having a higher number of Y-chromosome bearing sperm relative to X-chromosome bearing sperm.

In other embodiments of the invention, the window of time for separation or sexing is determined by locating a maximum in the curve obtained by plotting percent female cells determined by fluorescent in situ hybridization (FISH) against percent Koo positive cells, determining the time at which the maximum percent female cells occurs, and beginning the separation step no earlier than about one hour before the time of the maximum percent female cells.

In still other embodiments of the invention, the window is defined by time from collection of the sperm. Typically, the window is from about 2 to about 24 hours after collection of the sperm, preferably from about 2 to 12 hours, more preferably from about 4 to about 8 hours. Particularly, when the sperm is cooled promptly to about 12° C., the separation is performed at about 6 hours after collection of the sperm.

The opening and closing of this window can be affected by various factors. For example, cooling the semen to below room temperature after collection of the semen waiting for the window to open to provide effective separation can increase the waiting time to obtain maximum sex bias as determined by FISH. However, holding the semen at elevated temperature can delay the waiting time to maximum sex bias. In certain preferred embodiments of the invention, promptly cooling the semen to about 12° C. enables effective separation at about 6 hours after collection. Cooling tends to increases the maximum sex bias attainable relative to holding at room temperature. However, holding the semen at elevated temperature can diminish the extent of sex bias achievable. Changes in such factors can also affect the width of the window, i.e., the period during which the window is open, that time period being shorter at higher temperatures and longer at lower temperatures.

The invention further provides a method for separating a selected population of cells from a sample of semen, the method comprising cooling the semen to a predetermined temperature; waiting until a predetermined time for the sexing window to open; contacting the sample with a cell binding agent to preferentially bind to the selected population of cells for a time sufficient for the cell binding agent to bind the selected cells; and separating the selected cells and the cell binding agent to provide a treated sample containing non selected cells.

The present invention also provides methods for removing selected spermatozoa from a specimen of semen to provide a treated specimen having a higher percentage of sperm bearing the selected or preferred sex chromosome than in the original ejaculate. Preferred methods involve the steps of: collecting semen ejaculate; cooling the semen to a selected temperature; after a selected time, contacting the semen with a cell binding agent that preferentially selectively binds sperm bearing the non preferred sex chromosome, separating the cell binding agent with bound sperm bearing the non preferred sex chromosome from the remaining fluid, thereby providing a fluid containing a higher percentage of sperm bearing the preferred sex chromosome when compared to original ejaculate. The selected time is based, for example, on the appearance of sticky patches on the sperm or alternative criteria, as discussed in more detail herein. Typically, the ejaculate is cooled promptly to at least ambient temperature, preferably at least about 20° C., more preferably at least about 16° C., and even more preferably, at least about 12° C.

Thus, the invention also provides methods for increasing the probability of producing an offspring of a desired or preferred sex. For example, if bull semen is treated to bias the treated sperm to contain two-thirds viable X-chromosome bearing sperm and one-third viable Y-chromosome bearing sperm, twice as many female calves can be expected to be born than male calves when the treated semen is used to impregnate cows. The desired or preferred sex can vary with application. Also, the selected sperm cells can be either the bound cells or the non bound cells, depending on the application. Thus, the use of such terms as “preferred” or “desired” or “selected” are relative.

In certain preferred embodiments of the invention, the separating or fractionating step comprises incubating the ejaculate with a cell binding agent; permitting the sperm of a non preferred sex type to preferentially bind to the cell binding agent; and separating the cell binding agent with preferentially bound sperm of the non preferred sex type from non bound sperm, thereby providing a selected population of cells, the cell binding agent comprising a bead having a surface treatment comprising a material that preferentially binds to sperm of a non preferred sex.

In other preferred embodiments of the invention, the separating or fractionating step comprises incubating a mixture of the ejaculate with a cell binding agent; permitting the sperm of a non preferred sex type to preferentially bind to the cell binding agent; adding beads to the mixture, the beads being capable of binding with the cell binding agent; incubating the beads with the mixture to bind beads to the cell binding agent; and separating the beads and cell binding agent thereby preferentially removing sperm of the non preferred sex type from the mixture, preferentially leaving sperm of a preferred sex type.

As used herein, the term “Koo antibody” means antibody prepared by the procedures described by Koo, G. C. et al. in “Application of Monoclonal Anti-HY antibody for Human H—Y Typing,” Human Genetics, 1981, pp. 64-67, vol. 57, Springer-Verlag. Koo antibody is obtained preferably from hybridomas HB9070 or HB9071, which are available from the American Type Culture Collection.

As used herein, the term “Koo positive cells” means cells labelled with Koo antibody as examined microscopically after processing in a standard immunocytochemistry (ICC) protocol.

As used herein, the terms “X-bearing sperm” and “X-sperm” are used as a short hand notation for X-chromosome bearing sperm. Similarly, the terms “Y-bearing sperm” and “Y-sperm” are used as a short hand notation for Y-chromosome bearing sperm.

As used herein, the term “sticky patches” refers to the appearance, after immunocytochemistry, of a fluorescent punctate pattern on the upper portion of the sperm head as viewed under a microscope, for example at a a magnification of 400× (ocular of 10×, objective of 40×). The percent of sperm cells bearing sticky patches is determined as follows. The cell surface of washed sperm is conjugated with a primary sperm cell antibody and, subsequently, the primary antibody is conjugated to a fluorescently-labelled second antibody. A microscopist then scores sperm under the microscope for staining pattern. Using Koo as the primary antibody, the sperm stains in a punctate pattern on the anterior acrosomal membrane, in a fashion similar to what Wassarman has depicted for egg-binding proteins. (Paul M. Wassarman, Scientific American (1988), December, p. 83). The number of sperm cells exhibiting Koo staining increases with time, thus enabling a time window to be defined by lower and upper percentages of sperm in the population that react with the Koo antibody. Thus, the punctate pattern is capable of being detected and visualized using the Koo antibody, among others.

For example, few bull sperm are Koo-positive in freshly collected samples, at temperatures in the range of about 12 to 16° C., typically at least about 30% are positive at about 6 hours post collection, and most to almost all sperm are Koo positive by 24 hours post collection in neat ejaculates. Temperature appears to have a profound effect on Koo staining, both in terms of when staining appears and how much staining appears. Temperature conditions that impair staining or increase it to universal proportions are conditions that have been shown in experiments not to permit effective sexing (i.e., desired sex bias). Other sperm cell antibodies also can be used as primary antibodies for this work because our early experiments showed that many antibodies stain in the same pattern and timeframe as Koo. However, the definition of a window can change when other antibodies are used. The definition of a window also can change when spermatozoa of different species are used. In any event, the window can be determined by running simple tests as described and illustrated herein. This staining procedure also can be used to define a time window when other conditions for collecting and handling of mammalian semen (e.g., an ejaculate) are varied, to determine the effect of these conditions on the opening, breadth, and height of the window.

In a preferred embodiment of the invention, semen is collected at time zero, the temperature of the semen is reduced to 12° C. within about 30 minutes, and the semen is treated at about 6 hours to provide a fluid having a higher concentration of sperm cells bearing a desired sex chromosome.

### BRIEF DESCRIPTION OF THE DRAWINGS

FIG. 1 is a graph illustrating the increase in the percent of sperm cells ICC positive as a function of time at different temperatures.

FIG. 2 is a graph of % of cells positive with Koo antibody by ICC vs. the % of female cells determined by FISH at 4° C.

FIG. 3 is a graph of % of cells positive with Koo antibody by ICC vs. the % of female cells determined by FISH at 12° C.

FIG. 4 is a graph of % of cells positive with Koo antibody by ICC vs. the % of female cells determined by FISH at 28° C.

FIG. 5 is a graph of % of cells positive with Koo antibody by ICC vs. the % of female cells determined by FISH at 4° C. comparing data collected at two different times.

FIG. 6 is a graph of % of cells positive with Koo antibody by ICC vs. the % of female cells determined by FISH at 12° C. comparing data collected at two different times.

### DETAILED DESCRIPTION OF THE INVENTION INCLUDING PREFERRED EMBODIMENTS

The present invention provides methods for separation of X- and Y-bearing sperm which are competent (or viable) to fertilize mammalian eggs, e.g., eggs in fertile cows, using standard AI techniques currently employed on farm. As noted above, prior methods of separation often compromise sperm integrity, i.e., their motility and fertilization ability, so that fertilization utilizing such prior art separated sperm requires complicated techniques such as in vitro fertilization (IVF) or ultrasounding of cows during heat to determine side of ovulation, coupled with introduction of a low sperm dose by high uterine horn insemination into the horn attached to the ovary from which the egg is released. It is impossible to use these methods on farm with working dairy herds. The method of the invention can be utilized for separating X- and Y-sperm from a variety of mammalian species, including various livestock, such as cattle and sheep, as well as dogs, cats, horses, swine, and other species. The process also is applicable to humans.

By means of the present invention, a sperm sample containing a natural population of X- and Y-sperm can be treated to produce an X- or Y-enriched sperm subpopulation which is biased with respect to spermatozoa capable of producing the desired sex, i.e., the number of sperm functional for fertilization are sex-biased and the number of sperm bearing the desired sex chromosome is significantly increased with respect to the natural (i.e., untreated) semen. By “significantly increased,” for example, we mean that use of a treated semen sample enriched with X-sperm, when utilized for artificial insemination, can produce at least about 55% female offspring, preferealy, at least about 60% female offspring and, even more preferably, at least about 65% female oofspring.

Preferential separation of the X- or Y-spermatozoa is performed by treating the sperm cells during the sexing window. In a preferred embodiment of the invention, the sperm cells are treated with sperm cell binding agents during the sexing window, wherein Y-spermatozoa preferentially bind to the sperm cell binding agents and are removed with the sperm cell binding agents from the non bound spermatozoa. The non bound spermatozoa are then used to impregnate the fertile female mammal or to fertilize a suitable mammalian egg in vitro.

Preferred sperm cell binding agents comprise beads coated with a substance to which the sperm having sticky patches bind. Suitable substances for coating the beads include antibodies, proteins, charged and non charged molecules. Examples of such substances include lectins, IgM, IgG, not clear PrA binds these sperm!, diethyltriamine, glycine, polylysine, 1-(3-aminopropyl)-2-pipecoline, 3-aminopropylphosponic acid, 3-aminopropanesulfonic acid, DL-homocysteic acid, cyanoacetylurea, 3-aminopropionitrile fumarate, mercaptosuccinic acid, 3-mercapto-1-propanesulfonic acid sodium salt, 3-mercaptopropionic acid, 2-(aminoethyl)trimethylammonium chloride HCl, 2-(dimethylamino)ethanethiol HCl, Serinol, pentaethylene-hexamine, n-isopropyl-1,3-propanediamine, ethylamine, 2-(ethylthio)ethylamine HCl, 3-mercapto-1-propanol, 3-mercapto-1,2-propanediol, and the like. Such substances are bound to the beads using procedures well known to those skilled in the art. Beads can be formed of metal, glass or polymeric materials. Well known examples of beads are those sold under the trademarks Sephadex and Sepharose, and the like. Particularly, preferred are magnetic beads, e.g., beads containing magnetic materials (e.g., magnetic particles or a magnetic core) such that they can be separated from fluid media by using a magnetic field. Useful magnetic beads are those sold by VICAM, Watertown, Mass.

Also, antibodies which bind to X- or Y-specific proteins from sperm cells can be used for separation in accord with the present invention. These antibodies can be of any type of antibody (including IgG and IgM) and can be either polyclonal antibodies or monoclonal antibodies. If polyclonal antibodies are to be used, then such antibodies can be prepared according to per se known procedures. For example, procedures such as those described in Hurn, B. A. et al., (1980), Meth. in Enzymology, Ed. Van Vanakis, H. and Langone, J., pp. 104-142, can be used.

If desired, monoclonal antibodies can be utilized and prepared according to methods which are per se known in the art, such as those originally described by Milstein and Kohler, published in Nature (1975), 256, pp. 495-497. This basic procedure involves injecting an animal, usually a mouse, with an immunogenic substance. After suitable time for antibody production to the immunogen, the mouse is sacrificed. Cells are removed from the spleen and fused with myeloma cells. Hybridoma cells resulting from this fusion are able to reproduce in vitro, and each express genetic information for one specific antibody. The antibodies produced from one hybridoma fusion thus will tend to have high specificity.

Cells cultured from individual hybridoma cell lines can then be screened for production of antibodies to the desired target antigenic determinant. Those hybridomas positive for the target antigen can be further screened to identify those having the desired level of affinity. Monoclonal antibodies displaying all of these characteristics can then be screened using actual assay conditions to determine if the assay condition alters the antibody binding characteristics or affinity, and to screen out those with cross-reactivity to possible contaminating antigens.

In certain embodiments of the invention, preferred antibodies are those which are specific for and bind to Y-sperm, such as antibodies claimed to bind, for example, to the H-Y antigen. Such antibodies can be prepared, for example, by the procedure described in U.S. Pat. No. 4,680,258 to Hammerling et al.

Cell binding agents useful in the practice of this invention can be determined by performing ICC with the proposed cell binding agent to see whether a subpopulation of sperm binds to the proposed cell binding agent, as indicated by the cells in the bound subpopulation becoming fluorescent when the cell binding agent has a fluorescent label. This method can be useful for evaluating, for example, antibodies and proteins in general, including lectins. The method also is good for screening macromolecules as separating agents, so long as they can be labeled without loss or gain of function, or a labelled second antibody to them can be obtained. For small species such as, for example, sulfonate terminators on the beads, useful cell binding agents are best determined by a screening procedure where beads of the desired structure (i.e., having the desired coating or agent bound to the surface) are made and, then, after incubation and capture, one determines whether the beads clear a subpopulation of sperm from the supernatant (as opposed to all or none). Then, one determines whether the sperm remaining in the supernatant are sex-biased by assaying with a genetic method (e.g., FISH).

Antibodies specific for either X- or Y-spermatozoa can be immobilized on beads. These beads can be plastic beads or magnetic beads. Beads useful in the practice of certain embodiments of the invention are plastic beads, e.g., SEPHAROSE™ 6 MB, or other beads, that are large enough to be physically separated from the supernatant, e.g., to settle out in a separation or purification process or be captured by a magnetic field. When plastic beads are utilized, the beads having antibody bound thereto are mixed into a sperm sample and, subsequently, separated from the bulk fluid, e.g., allowed to settle to the bottom of the container. This step can be repeated, if desired, to increase the completeness of separation of sperm according to sex chromosome. Centifugal forces or magnetic fields, etc. can be used to facilitate the separation. Care should be taken to avoid excessive forces that would harm or incapacitate the desired spermatozoa.

If magnetic beads are used, the beads preferably are microspheres of magnetic particles representing an immobilizing matrix. It has been found that magnetic beads having a diameter of from 0.1 to 2 microns in diameter can be particularly useful in certain embodiments of the present invention for separating the desired species of spermatozoa without compromising the motility and fertilization ability of the spermatozoa. Particularly useful magnetic beads are described, for example, in U.S. Pat. No. 5,071,076; U.S. Pat. No. 5,108,933; U.S. Pat. No. 4,795,698; and PCT Patent Publication No. WO91/09678. According to the procedures described in these patents, preferred beads can be prepared having a diameter of 0.1 to 0.5 microns.

Antibodies can be bound to the beads by means of procedures which are per se known in the art. In general, a linking compound is attached to the beads during manufacture of the beads. After the antibody is bound to the beads, the beads are washed so only attached antibody remains. Additional procedures known to those skilled in the art are described, for example, in U.S. Pat. No. 4,018,886; U.S. Pat. No. 3,970,518; U.S. Pat. No. 4,855,045; and U.S. Pat. No. 4,230,685. Protein A is a preferred linking compound which greatly increases the effectiveness of binding of the antibodies. (See, Forsgren et al. (1977) J. Immunol. 99: 19, the disclosure of which are hereby incorporated by reference.) Protein A attaches to the Fc portion of IgG subclass antibodies, thus extending and presenting the Fab portion of these antibodies. The resulting correct orientation of the antibodies and extension away from the particles leads to a very effective interaction between the bound antibodies and their target.

The method of attachment of Protein A to magnetic particles can proceed by any of several processes available through and described in known scientific literature. In one such procedure, magnetic iron oxide particles of approximately one micrometer diameter are chemically derivatized by a reaction, first with 3-aminopropyltri-ethoxysilane, then with glutaraldehyde. The derivatized magnetic particles are then mixed with Protein A resulting in a magnetic particle to which Protein A is covalently attached. The antibodies are then added to the Protein A magnetic particles and after a short incubation, the Protein A-antibody complexes form. (See, Weetall, H. H. (1976) Meth. In Enzymol. 44:134-48.)

In one embodiment of the present invention, a magnetic separator is used for magnetically separating different components of a test sample. Generally, the test sample can be prepared as a mixture of magnetic components and non-magnetic components. For example, during the sexing window, coated magnetic beads can be used as sperm cell binding agents to preferentially bind Y-sperm. The magnetic component can be manipulated or controlled within the mixture by the application of a magnetic field. Thus, the magnetic component can be separated from the non magnetic component to allow a user to select one or both of the components for a desired property or properties, e.g., for use in artificial insemination of mammals in an effort to produce offspring of a desired sex.

The magnetic separator employs a magnet that provides a magnetic field to separate the magnetic components from the non-magnetic components. The magnet can be configured to control movement of the magnetic components toward a desired region of the separator and away from the non-magnetic components. Once separated, the non-magnetic components readily can be removed from the magnetic separator substantially separate from the magnetic components with minimal remixing of the non-magnetic and magnetic components.

The magnetic separator can include a container-receiving region that is configured to receive a container for holding the test sample. The magnet can be arranged in the separator to produce an external magnetic field within the container-receiving region of the separator so as to act upon the test sample in the container. A guide can be provided to position the container at a predetermined location within the container-receiving region relative to the magnet to subject each test sample to a consistent magnetic field. One or more retainers can be provided to hold the container in a desired position relative to the magnet.

The separator can include one or more magnets that are positioned at predetermined locations about the container-receiving region to produce a desired magnetic field. For example, the separator can utilize a dipole arrangement in which a pair of magnets are positioned on opposite sides of the container-receiving region approximately 180° apart. However, any suitable magnet arrangement, such as three or four (quadrapole) equally spaced magnets or multiple non-equally spaced magnets, can be incorporated in the separator to provide the desired affect. Each magnet can be a bar magnet formed from any suitable magnetic material. Also, other suitable magnetic sources or generators, such as an electromagnet, can be utilized for the magnet.

The magnetic separator can include a container that is configured to hold the test sample within the magnetic field during separation, and then allow the non-magnetic components to be drawn off once separated. In this regard, the container can have an inlet for receiving a test sample and an outlet through which the non-magnetic components can be released after separation. The outlet can be positioned at any convenient location, preferably at a lower portion of the container to allow gravitational flow from the container. Additionally, the non-magnetic components can be removed from the container using any suitable device, such as a pump.

A regulator can be coupled to the outlet of the container to regulate the flow of the non-magnetic particles from the container. The regulator can be any suitable device to regulate the flow from the outlet, including a clamp or a valve, e.g., a stopcock.

The magnetic separator can be particularly suitable for separating a test sample having relatively high concentrations of magnetic or non-magnetic components. In this regard, the test sample can be held within the magnetic field by the container for a sufficient period of time to allow separation of the components. Once separated, the regulator can be actuated to release the non-magnetic components from the container at a controlled rate that reduces the likelihood that magnetic components will remix and be drawn from the container along with the non-magnetic components.

The magnetic separator can include a base that supports the container-receiving region above a vessel-receiving region that is configured to receive a vessel for capturing the non-magnetic components released from the container.

To ensure adequate separation of the test sample has occurred prior to release of the non-magnetic components, the container can include a window or be formed of a see-through material to allow a user to visually monitor the amount of separation. Once the test sample is separated, the user also can monitor the test sample as the non-magnetic components are being withdrawn from the container to reduce the likelihood that magnetic components are inadvertently released from the container along with the non-magnetic components.

A particularly useful magnetic separator is described in copending application Ser. No. 10/346,576 filed Jan. 16, 2003 and commonly assigned with the present application, the the disclosure of which are hereby incorporated by reference.

In one embodiment of the invention, the method includes the step of contacting the biological sample in the sexing window with a magnetic cell binding agent (e.g., a cell binding agent immobilized on a magnetic bead) that preferentially binds the desired population of spermatozoa to form a magnetic component of the biological sample. A magnetic field is then applied to the binding agent with bound spermatozoa (e.g., using a magnetic separation device) to separate the magnetic component from the non-magnetic components of the biological sample. The non-magnetic components of the biological sample then are removed from the container to separate the non bound population of cells from the magnetic components of the biological fluid sample. Typically, the removal of the non-magnetic components from the container is by draining the non-magnetic components out of the bottom of the container.

The cell binding agent can be a molecule or moiety that is either linked to the magnetic particle or not linked to the magnetic particle when it added to the biological sample. When unlinked cell binding agents are used, the cell binding agent is contacted with the biological sample for a time sufficient to bind the selected population of cells. A magnetic particle containing a linking compound is subsequently added to bind the cell binding agent (with bound cells) to the magnetic particle. For example, the linking compound can be an antibody to the cell binding agent molecules. Various combinations of linking compounds (or “linking agents”) and cell binding agents are well known from antibody and double antibody tests well known to those skilled in the art.

Preferably, the removal of the non-magnetic components from the container is performed by draining the non-magnetic components out of the bottom of the container. An important feature of draining the non-magnetic components from the container, rather than removing these components by aspiration, is that aspiration tends to mix the liquid by creating vortices and other turbulent fluid movement. In methods of cell separation, particularly in which the magnetic field applied does not necessarily hold the magnetized component immobile against container walls, it is important to keep turbulent fluid movement to a minimum. This preferably is achieved by removing fluid from the bottom of the container, using laminar flow, which limits remixing of the sample components. Thus, in a preferred embodiment, the step of separating the selected population of cells from the magnetically held cells is performed by draining the container by gravity. Other methods for draining the container, such as by pump or regulated pressure also can be used. In a typical application, the step of draining is regulated by opening and optionally closing a valve or stopcock to regulate the flow of the non-magnetic components from the container. If a pump is used, then regulating the operation of the pump attached to a drain of the separator container will achieve the same effect.

Another method for removing the selected population of cells from the container without disturbing the cell separation includes pumping a dense fluid inert to sperm (i.e., denser than the non-magnetic components to be removed) into the container to displace the non-magnetic components of the biological sample from the container. Using this method, the non-magnetic components can be removed from the top of the container rather than by draining from the bottom.

A feature of the use of the magnetic separator is that separated populations of cells can be recovered from the device after separation with little waste. For example, in accord with one preferred embodiment of the invention, one can fractionate the sperm cells by binding Y-bearing spermatozoa preferentially to magnetic particles during the sexing window. Thus, the magnetic separator “pulls” out the Y-bearing sperm bound to the particles and X-bearing sperm (the non-magnetic component of the biological sample) can be drained out of the magnetic separator. The magnetic separator is constructed preferably to permit all but a small amount of the non-magnetic component to be removed from the separation container; the small amount is left behind to ensure that the desired population of cells is recovered. In operation this is similar to removing the bottom phase in a separatory funnel. After removing the non-magnetic components, the small amount of non-magnetic components is removed (similar to the interface between phases in a separatory funnel). This leaves the magnetic components in the separation container, i.e., the cells that are bound to the cell binding agent (e.g., antibody-linked or coated beads). These bound sperm cells also can be recovered, thus, providing separation and isolation of the two populations of spermatozoa (i.e., X- and Y-bearing sperm). Recovery of the magnetic component is easily performed by removing the separation container from the magnetic separator and then draining the container. Reversal of the binding is accomplished in the same manner as in certain types of chromatography or antibody-antigen reactions.

The separation methods can be repeated sequentially on a single population of cells to further purify the cells (e.g., using the same or a different binding agent that also recognizes the cells or a subpopulation thereon), or can be repeated sequentially on a mixed population of cells using cell binding agent molecules that bind to different populations of cells in order to recover several different populations of cells.

The binding agents can be of any kind that bind cells with sufficient affinity and/or avidity to remain bound during the separation procedures. Exemplary binding agent molecules include antibodies, lectin molecules, phage display molecules (or other combinatorial binding molecules), binding partners of a cell surface molecule (e.g., one of a ligand-receptor pair such as CD4-CD4 receptor; a carbohydrate or carbohydrate-containing molecule (such as a glycoprotein) and a carbohydrate receptor on the cell surface), etc.

As is well-known in the art, only a small portion of an antibody molecule, the paratope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). Within the antigen-binding portion of an antibody, there are complementary determining regions (CDRs), which directly interact with the epitope of the antigen, and framework regions (FRs), which maintain the tertiary structure of the paratope (see, in general, Clark, 1986; Roitt, 1991). It is now well-established in the art that the non-CDR regions of a mammalian antibody may be replaced with similar regions of conspecific or heterospecific antibodies while retaining the epitopic specificity of the original antibody. See, e.g., U.S. Pat. Nos. 4,816,567, 5,225,539, 5,585,089, 5,693,762 and 5,859,205.

Accordingly, cell binding agents useful in the practice of this invention also can comprise polypeptides of numerous size and type that bind specifically to cell-surface molecules, including polypeptides, carbohydrates, lipids, and combinations thereof. These polypeptides can be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful in the practice of the present invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to a particular cell type that is to be separated using the magnetic separator device. This process can be repeated through several cycles of reselection of phage that bind to the particular cell type. Repeated rounds lead to enrichment of phage bearing particular sequences. DNA sequence analysis can be conducted to identify the sequences of the expressed polypeptides. The minimal linear portion of the sequence that binds to the particular cell type can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the particular cell type.

In preferred embodiments, antibodies and other cell binding agent molecules can be bound to particles (e.g., beads or preferably magnetic beads) using procedures which are well known to the person of ordinary skill in the art. Antibodies and other cell binding agent molecules can be covalently linked directly to the particles, or can be attached to the particles through an intermediate linking compound. A linking compound can be attached to the beads during manufacture of the beads. An antibody or other cell binding agent for the spermatozoa is bound to the beads by the linking compound. For example, preferred magnetic beads can be prepared by mixing beads at about 1 mg iron/ml with purified antibody at 1 mg/ml protein. After the antibody is bound to the beads, the beads are washed so that only attached antibody remains. Additional procedures known to those skilled in the art are described, for example, in U.S. Pat. No. 4,018,886; U.S. Pat. No. 3,970,518; U.S. Pat. No. 4,855,045; and U.S. Pat. No. 4,230,685.

Examples of an intermediate linking compound for antibodies include Protein A, Protein G, and other proteins that specifically bind antibodies, lectins, receptors and the like, including antibodies that bind other antibodies, such as anti-Fc antibodies, anti-IgG antibodies or anti-IgM antibodies. Protein A is a preferred linking compound which greatly increases the effectiveness of capture. (Forsgren et al., (1977) J. Immunol. 99:19(This is totally redundant, unless you really want in in here.)

The method of attachment of Protein A to the beads can proceed by any of several processes well known to those skilled in the art. In one such procedure, magnetic iron oxide particles of approximately one micrometer diameter are chemically derivatized by a reaction, first with 3-aminopropyltriethoxysilane, then with glutaraldehyde. The derivatized magnetic particles are then mixed with Protein A resulting in a magnetic particle to which Protein A is covalently attached. The antibodies are then added to the Protein A magnetic particles and after a short incubation, the Protein A-antibody complexes form (see, Weetall, H. H. (1976) Meth. Enzymol. 44:134-48).

Magnetic particles useful in the practice of this invention preferably are non-porous magnetic beads. Preferably, the diameter of the beads is less than about 10 microns, more preferably less than about 5 microns. The particular bead magnetic particles that provide an optimal recovery of a desired population of cells can be selected by one of ordinary skill in the art by testing beads of different sizes and properties using a particular magnetic separator that will be used to carry out the methods of the invention. In particularly preferred embodiments, the magnetic beads have a diameter of 0.1 to 2 microns, and more preferably have a diameter of 0.1 to 0.5 microns. Additional useful magnetic beads are described, for example, in U.S. Pat. No. 5,071,076; U.S. Pat. No. 5,108,933; U.S. Pat. No. 4,795,698; and PCT Publication No. WO91/09678.

The methods for cell separation using a magnetic device, in accord with certain embodiments of the invention, are particularly useful for separating large populations of cells such as when the biological sample contains greater than about 1×10⁵ cells, greater than about 1×10⁶ cells, greater than about 1×10⁷ cells, greater than about 1×10⁸ cells, greater than about 1×10⁹ cells, or more.

The ability to separate—efficiently, quickly and in a way that preserves their integrity—a large number of cells permits the economic separation of cells for artificial insemination applications, particularly for agricultural uses, in which multiple ejaculates must be processed to service large insemination operations. Thus, the separation of spermatozoa from animal ejaculates into spermatozoa preferentially determinative of one sex is a preferred use of the magnetic separator device. The ability to separate efficiently a large number of cells also permits the separation of whole ejaculates, without discarding any of the desired type of spermatozoa. Thus, whole ejaculates can be used efficiently in contrast to existing methods in which portions of desired spermatozoa are discarded or wasted in the processing procedure.

The efficiency and gentleness of the cell separation using the magnetic separator provides opportunities for methods of artificial insemination in which a population of spermatozoa obtained using the magnetic separator is used to inseminate a mammal. Standard methods of artificial insemination that are well known in the art can be used, including combining separated spermatozoa with standard extension composition (e.g., including egg yolk and various other components), packing separated spermatozoa into straws and optionally freezing and storing them, and inseminating animals with the separated spermatozoa. It is even possible to use the magnetic component of the separation methods without further purification from the magnetic particles, i.e., a selected population of cells that are bound to magnetic particles, by inseminating a fertile mammal with the sperm cells bound to the particles.

Therefore, the present invention provides methods of increasing the percentage of mammalian offspring of either sex. The methods include preferentially separating spermatozoa determinative of one sex from a biological sample containing spermatozoa determinative of both sexes by carrying out the methods described herein. Once the spermatozoa preferentially determinative of one sex are separated from the remainder of the biological fluid sample containing the spermatozoa preferentially determinative of the other sex, either population of separated spermatozoa can be administered to the reproductive tract of a female animal, preferably a mammal, preferably using artificial insemination techniques. Further steps, such as washing the isolated and separated spermatozoa prior to administering the spermatozoa to the reproductive tract of a female animal also can be performed. As used herein, “mammal” includes cattle, sheep, pigs, goats, horses, dogs, cats, primates or other mammals.

If not constrained by loss of sperm integrity during sexing, artificial insemination techniques can use either “high dose” or “low dose” methods (reflecting the relative amounts of spermatozoa used for insemination; the methods of the invention are applicable with any amount of spermatozoa (i.e., including both high dose and low dose methods). In certain embodiments of the methods using spermatozoa separated using the methods of the present invention, a relatively high dose is used, e.g., greater than about 10 million cells are used for insemination. In these embodiments, the number of spermatozoa administered preferably is at least about 20 million, more preferably at least about 30 million, still more preferably at least about 40 million, and yet more preferably at least about 50 million. In other embodiments of the methods using spermatozoa separated using the methods of the present invention, a relatively low dose is used, e.g., less than about 10 million cells are used for insemination. In these latter embodiments, the number of spermatozoa administered preferably is less than about 5 million, more preferably is less than about 1 million and still more preferably is less than about 0.5 million.

The use of the magnetic separator, because it can efficiently and gently separate large numbers of cells with low cell loss, provides the ability to fractionate an entire ejaculate of a mammal in a single process, which is not achievable using current methods of cell separation such as fluorescence activated cell sorting (FACS). FACS typically rejects greater than 90% of the input cells and causes damage to the sorted ones. The ability of the methods of the invention to separate large numbers of cells with low cell loss and preservation of cell integrity is an advantage for artificial insemination operations and other organizations that process many ejaculates. The ability to fractionate entire ejaculates is advantageous even for smaller organizations and individual farmers that may separate spermatozoa only for their own herd.

In a preferred embodiment of the invention, to fractionate an entire ejaculate, it is combined with magnetic particles coated with a binding agent and, then, subjected to separation using the magnetic separator as described above. The ejaculate in some embodiments is fractionated with an effectiveness that provides at least about 55% of the desired sperm sex type in the separated component, preferably at least 60%, and more preferably at least 65%.

Subsequent to fractionating the ejaculate, animals (preferably mammals) can be inseminated with the population of spermatozoa preferentially determinative of the one desired sex. Because the methods of fractionation and cell separation using the magnetic separator are efficient and gentle to cells that are easily damaged, such as spermatozoa, most of the cells isolated using the methods retain their activity as compared to unfractionated cells. For spermatozoa, this means that conception rates for animals inseminated with fractionated cells are maintained at levels similar to that using unfractionated cells. In contrast, prior methods of cell separation often compromise the motility and fertilization ability of spermatozoa due to the use of harsh conditions including exposure to laser light and dye molecules (FACS), shear forces, etc., so that fertilization utilizing such separated spermatozoa requires complicated and expensive techniques and lowers the efficiency of conception. Further, such techniques are not suitable for use on a farm. Thus, using a magnetic separator, in accord with a preferred embodiment of the invention, to separate spermatozoa that are then used in standard insemination procedures, the conception rate of offspring resulting from the insemination is, in preferred embodiments at least about 50% of the conception rate obtained using unfractionated spermatozoa. In more preferred embodiments, the conception rate is higher and approaches that seen using unfractionated spermatozoa (e.g., at least about 70%, 80%, 90%, or 95% of the conception rate obtained using unfractionated spermatozoa). These methods, therefore, are useful for creating a sex bias in mammalian offspring without the use of IVF, embryo transfer or other expensive procedures.

Another feature of the separation using the magnetic separator in accord with a preferred embodiment of the invention is the ability to quickly fractionate large numbers of cells, which is particularly useful for separation of cells where biological activity must be retained as much as possible. For example, an entire ejaculate can be fractionated in less than about 2 hours. Preferably an entire ejaculate is fractionated in less than about 1 hour. This can be contrasted with FACS methods that require many more hours to process large numbers of cells at low yield (e.g., about 7-10 straws per day), thereby exposing the cells to dye compounds for long times and long storage times while awaiting fractionation. The ability to process ejaculate in short times facilitates the processing of large quantities within the sexing window in accord the present invention.

By using the magnetic separator in accordance with the preferred methods described herein, spermatozoa of a mammal can be fractionated quickly and without a substantial loss of quality. Quality includes, but is not limited to: motility, progressive motility, grade of motility, acrosomal integrity, immediate and incubated post-thaw motility and morphology. Thus, the quality of the fractionated spermatozoa using these methods is at least about 50% of the unprocessed spermatozoa. Preferably, the functionality of the fractionated spermatozoa is at least about 60% of the unprocessed spermatozoa, at least about 70% of the unprocessed spermatozoa, at least about 80% of the unprocessed spermatozoa, or is at least about 90% of the unprocessed spermatozoa. More preferably, the quality of the fractionated spermatozoa is at least about 95% of the unprocessed spermatozoa, still more preferably is at least about 97% of the unprocessed spermatozoa, yet even more preferably is at least about 98% of the unprocessed spermatozoa, and most preferably is at least about 99% of the unprocessed spermatozoa. Populations of fractionated spermatozoa preferentially determinative of one sex having the foregoing levels of quality relative to unprocessed spermatozoa are provided.

Thus, methods for fractionating ejaculates in the sexing window can use any of various known separation technologies but, preferably, uses a magnetic separation technology. The invention, therefore, provides populations of separated spermatozoa of good quality and having a bias for producing offspring of a desired sex, methods for artificial insemination using such populations of spermatozoa, and other methods and products that are described more fully herein.

The invention will be described further in the following examples. In the examples that follow, the following methods, procedures and media are used. Matured bovine oocytes were purchased from Trans Ova Genetics (2938 380^{th} St., Sioux Center, Iowa 51250).

General Procedures

Procedure 1. Sperm Treatment Procedure (Swimming Up Procedure)

1. Two 0.25 ml straws of frozen semen (fractionated or unfractionated) were thawed for 10 sec at 37° C.
2. Five 100 μl aliquots of thawed semen were layered in 12×75 mm polypropylene round-bottom tubes under 1 ml aliquots of the modified Tyrode's albumin lactate medium (mTALP) containing 6 mg/ml fatty acid free bovine serum albumin (BSA; Sigma), 10 mM sodium pyruvate, and 50 μg/ml gentamycin.
3. After 1 h incubation at 38.5° C., the top 800 μl of medium (containing swim up spermatazoa) from each tube was taken and pooled in a 15 ml conical centrifuge tube.
4. The swim-up spermatozoa were then diluted with swim up medium to a final volume of 10 ml, centrifuged at 2,000 rpm (900×g) for 10 min, and the supernatant was discarded.
5. The sperm pellet was diluted to 10 ml with swim up medium, centrifuged again at 2,000 rpm (900×g) for 10 min, and 100 μl of sperm pellet was isolated.
6. The concentration of sperm in the pellet was then determined and the sample diluted to 1×10⁶ spermatozoa/ml with fertilization medium.
   Procedure 2. Fertilization In Vitro (IVF)
1. Make a 50 ul fertilization droplet using fertilization medium containing 1×10⁶ spermatozoa/ml (from above step 6) in a 35×10 mm cell culture dish.
2. Wash matured oocytes three times with wash medium, and then transfer 10 washed oocytes to each above fertilization droplet.
3. Place the cell culture dishes in an incubator with a 5% CO₂ atmosphere at a temperature of 38.5° C.
   Procedure 3. Fertilized Oocyte Culture In Vitro
1. Make a 50 μl droplet using Developmental medium I in 35×10 mm cell culture dishes. Fertilized oocytes were washed by Developmental medium I and transferred to those dishes containing 50 μl droplets of Developmental medium 1. Mineral oil is added to completely cover the separated droplets containing eggs.
2. Five days later, Developmental medium I was replaced by Developmental medium II, by aspirating half of the original medium and adding fresh medium
3. At day 8 after fertilization, different stages of embryos including blastocsyts, morula, and developmentally arrested 8-16 cell embryos were collected for sexing.
   Procedure 4. Embryo Sexing

Embryo sexing was performed using the embryo PCR protocol as described in the Journal of Reproduction and Fertility (1993) 98, 335-340, with aminor modification.

All embryos (from 8-cell to hatched blastocyst stage) were produced by in vitro fertilization (IVF). A few embryonic cells are extracted from these embryos and put into a 250 μl PCR tube containing 5 μl lysis buffer (2% 2-mercaptoethanol; 0.01% SDS (sodium dodecylsulfate); 10 mM EDTA (ethylenediamine tetraaceic acid); 10 mM Tris-HCl, pH 8.3; proteinase K, 222 μg/ml). All embryonic cells were lysed at 55° C. for 2 hr, and proteinase K was inactivated at 98° C. for 10 min. Then, the sample was ready for PCR sexing.

The first round PCR was done using primers complementary to both ZFX and ZFY genes:

(forward primer: ATAATCACATGGAGAGCCACAAGCT)
(reverse primer: GCACTTCTITGGTATCTGAGAAAGT);

the nested PCR was used to specifically amplify the ZFX or ZFY gene using the allele-specific primers:

for ZFX:
forward primer: GACAGCTGAACAAGTGTTACTG
reverse primer: AATGTCACACTTGAATCGCATC
for ZFY:
forward primer: GAAGGCCTTCGAATGTATAAC
reverse primer: CTGACAAAAGGTGGCGATTTCA).

The primers used for nested PCR were to amplify non-overlapping regions of either ZFX or ZFY gene, and to generate 247 base pair (bp) (ZFX) and 167 bp (ZFY) products. PCR reactions consisted of 1× GeneAmp® PCR Gold buffer (15 mM Tris-HCl, pH 8.0; 50 mM KCl), 2.5 mM MgCl₂, 45 MdNTP for each dNTP, 250 nM of each primer and 1 unit of AmpliTaq Gold DNA polymerase (Applied Systems, N.J.) in a 50 μl reaction volume.

The first round PCR was done by hot-start at 94° C. for 10 min and 5 cycles of denaturation at 94° C. for 1 min, annealing at 55° C. for 1 min and extension at 72° C. for 1 min, and followed by 25 cycles of denaturation at 94° C. for 20 seconds, annealing at 55° C. for 20 seconds, extension at 72° C. for 30 seconds, and final extension at 72° C. for 10 min. 2 μl of the first round PCR products were used for the nested PCR, ZFX and ZFY were amplified in separat tubes, and the cycling protocol was performed in two stages with different annealing temperatures. The annealing temperature of the first five PCR cycles was 52° C., and the remaining 25 PCR cycles was 60° C. The nested PCR was also done by hot-start at 94° C. for 10 min, with a total 30 triphasic cycles of denaturation at 94° C. for 1 min, annealing at the temperature described above for 45 seconds and extension at 72° C. for 1 min and with a final extension for 5 min.

Following amplification, 7 μl of PCR products were mixed with 2 μl of loading buffer (20% Ficoll 400; 1% SDS and 0.25% Xylene Cyanol in 0.1M Na₂EDTA, pH=8) and loaded onto 1.5% (W/V) NuSieve™ agrose Gel containing Ethidium bromide (0.5 μg/ml), and resolved in Tris-acetate EDTA buffer by electrophoresis for 45 min at 82 V, and visualized by an UV transilluminator mounted with camera.

Procedure 5 S-Bead Semen Sexing Protocol (1.0 ml; Separation Using S-Magnetic Beads)

I. Sperm Cell Preparation
1. Collect ejaculate and immediately transfer raw ejaculate to a clean 15 ml plastic disposable centrifuge tube. Place the tube containing the sample into a 250 ml beaker containing 200 ml of water warmed to 32° C. and immediately place the beaker into a cooling bath set at 12° C. It is preferred to begin cooling the ejaculate as quickly as is practical post ejaculation. Commencing cooling more than 2 minutes post ejaculation can have a deleterious effect on the sexing results.
2. Record the Volume of Ejaculate Used on line 9 of the data sheet determine the Initial Cell Count (this method is for 1.0 ml samples of raw ejaculate)—. Record the Initial Cell Count of the ejaculate on line 12 of the data sheet. Determine the % Motility of the raw ejaculate and enter on line 11 of the data sheet.
3. Extend and pack the remainder of the semen sample as per normal procedure. Following packing immediately prepare the control straws for use for FISH analysis. Record the Volume of Ejaculate Extended as Control on line 10 of the data sheet.
4. Calculate the Total Initial Cells by multiplying the Volume of Ejaculate Used (line 9) by the Initial Cell Count (line12) and enter the result on line 13.
5. Cool the semen sample at 12° C. for 6 hours prior to continuing with Section III. (0 hr=ejaculation time)
   II. Cell Washing
1. Following the 6 hour cooling process transfer a 1.0 ml sample to a clean 15 ml conical tube and using the markings on the side of the tube, dilute the sample up to 4 ml with PBS at ambient temperature (generally about 15° C. to about 30° C.; preferably, about 20 to 25° C.). Mix by gently inverting the tube.
2. Pellet the spermatozoa by centrifugation of the sample at 900×g for 10 minutes at room temperature. Carefully aspirate and discard the supernatant. Remove as much of the supernatant as possible without disturbing the pellet, Semen pellets can be very soft and as much as 1.0 ml of liquid may remain in the tube following aspiration. Re-suspend the pellet, using the calibration markings on the side of the centrifuge tube, up to 4.0 ml with PBS.
3. Pellet the spermatozoa a second time by centrifugation at 900×g for 10 minutes at room temperature. Carefully aspirate and discard the supernatant. Re-suspend the pellet, using the calibration markings on the side of the centrifuge tube, to a final volume of 3.0 ml by adding PBS.
   III. Capture and Separation
1. Transfer 1.0 ml (concentration is 1.0 mg iron/ml in PBS) of S-Magnetic Beads (Surface-modified beads, which would include, for example, magnetic beads having a sulfonate treated surface; or beads having another desired surface treatment) to a 4 ml Cryovial and allow the magnetic beads to migrate to the side of the vial for 5 minutes in a Cryovial Magnetic Separator. (Surface-modified magnetic beads, vials and magnetic separator are available from Vicam, Watertown, Mass.) After five minutes in the magnetic separator the supernatant should be clear. Remove the supernatant and re-suspend the beads with 1.0 ml of PBS. Repeat the separation and re-suspension of the S-Magnetic beads a second time for a total of two washes. Following the second wash completely re-suspend the magnetic bead pellet by vortexing vigorously.
2. Add 600 μl of the washed re-suspended beads directly to the washed cell suspension prepared in Section III step 3 and completely mix by gently inverting the tube.
3. Incubate the cell/bead reaction mixture at room temperature for 30 minutes on a rotator at 4 RPM (Cole Parmer Roto-Torque Model 7637 or equivalent).
4. After incubating the cell/bead reaction mixture for 30 minutes transfer the cell/bead suspension to a 4 ml cryovial mounted in the cryovial magnetic separator and allow the magnetic beads to pull to the side of the vial for 10 minutes.
5. After pulling the bead-bound cells to the side of the cryovial for ten minutes carefully aspirate the supernatant from the cryovial while the vial remains fixed in the magnetic separator. Careful aspiration of the sample will minimize shifting and re-dispersion of the collected magnetic bead pellet. Do not collect any of the bead-bound portion of the solution.
6. Extend and freeze the collected sample according to normal freezing procedures.

Magnetic beads used in the above procedures and examples below were obtained from VICAM (Watertown Mass.). The beads also can be prepared by techiques well known to those skilled in the art.

When magnetic beads are used, the beads preferably are microspheres of magnetic particles representing an immobilizing matrix. It has been found that magnetic beads having a diameter of from 0.1 to 2 microns in diameter are specifically useful for separating the desired types of spermatozoa without compromising the motility and fertilization ability of the spermatozoa. Particularly useful magnetic beads are described, for example, in U.S. Pat. No. 5,071,076; U.S. Pat. No. 5,108,933; U.S. Pat. No. 4,795,698; and PCT Pat. No. Publication No. W091/09678. According to the procedures described in these patents, beads can be prepared having especially a diameter of 0.1 to 0.5 microns.

The antibodies are bound to the beads by means of procedures which are per se known to those skilled in the art. In general, a linking compound is attached to the magnetic beads during manufacture of the beads. A preferred linking compound is Protein A. On to the beads, an antibody (such as an IgG antibody which is directed against mouse IgM) is bound preferably by mixing beads in PBS at about 1 mg iron/ml with purified antibody reconstituted to the manufacturer's specifications at 1 mg/ml protein. After the antibody is bound to the beads, the beads are washed so that only attached antibody remains. Additional procedures known to those skilled in the art are described, for example, in U.S. Pat. No. 4,018,886; U.S. Pat. No. 3,970,518; U.S. Pat. No. 4,855,045; and U.S. Pat. No. 4,230,685. Protein A is a preferred linking compound because it greatly increases the effectiveness of capture by the attached antibodies. (Forsgren et al., (1977) J. Immunol. 99:19). Protein A attaches to the Fc portion of IgG subclass antibodies, thus extending and presenting the Fab portion of these antibodies. The resulting correct orientation of the antibodies and extension away from the particles leads to a very effective interaction between the bound antibodies and their target.

The method of attachment of Protein A to magnetic particles can proceed by any of several processes available through known scientific literature. In one such procedure, magnetic iron oxide particles of approximately one micrometer diameter are chemically derivatized by a reaction, first with 3-aminopropyltriethoxysilane, then with glutaraldehyde. The derivatized magnetic particles are then mixed with Protein A resulting in a magnetic particle to which Protein A is covalently attached. The antibodies are then added to the Protein A magnetic particles and after a short incubation, the Protein A-antibody complexes form. (Weetall, H. H. (1976) Meth. In Enzymol. 44:134-48).

In further example, to make Koo beads, VICAM magnetic beads were mixed at a concentration of 1 ml particles at 1 mg iron/ml with 1 ml of 1 mg/ml rabbit anti-mouse IgM antibody reconstituted per manufacturer's instructions (Bethyl Laboratories, Montgomery, Tex.). This mixture was rotated for 2 h at about 4 rpm on a Roto-Torque model 7637-01 (Cole-Parmer Instrument Company, Chicago, Ill.). Beads were captured magnetically for 5 min using the rack described above, and washed 4 times with 2 ml PBS. Beads were resuspended in 2 ml PBS, at which point they were mixed with 2 ml Koo antibody HB9070 (from ascites at about 2 mg IgM/ml) and rotated for 2 h at 4 rpm as described. Beads were then magnetically captured and washed 4 times with 4 ml PBS and resuspended in 2 ml PBS.

Procedure 6 Chase Capture Semen Sexing Protocol

I. Sperm Cell Preparation
1. Collect ejaculate and immediately transfer raw ejaculate to a clean 15 ml plastic disposable centrifuge tube. Place the tube containing the sample into a 250 ml beaker containing 200 ml of water warmed to 32° C. and immediately place the beaker into a cooling bath set at 12° C.
WARNING: The ejaculate sample must begin the cooling process as quickly as is practical post ejaculation. Under no circumstances should cooling commence more than 2 minutes post ejaculation. Delay in the cooling step may result in failure of the separation.
2 Cool the semen sample at 12° C. for 6 hours prior to continuing with Section II (0 hr=ejaculation time)
   II. Cell Washing
1. Following the 6 hour cooling process transfer a 1.0 ml sample to a clean 15 ml conical tube and, using the markings on the side of the tube, dilute the sample to 4 ml with PBS. Mix by gently inverting the tube.
2. Pellet the spermatozoa by centrifugation of the sample at 900×g for 10 minutes at room temperature. Carefully aspirate and discard the supernatant. Remove as much of the supernatant as possible without disturbing the pellet. Semen pellets can be very soft and as much as 1.0 ml of liquid may remain in the tube following aspiration. Re-suspend the pellet, using the calibration markings on the side of the centrifuge tube, to 4.0 ml with PBS.
3. Pellet the spermatozoa a second time by centrifugation at 900×g for 10 minutes at room temperature. Carefully aspirate and discard the supernatant. Re-suspend the pellet, using the calibration markings on the side of the centrifuge tube, to 3.0 ml with PBS.
   III. Primary Antibody Labeling of Sperm Cells
1. For each ml of sperm cells prepared in Section II, add 30 μl of a 2.0 mg/ml solution of primary sexing antibody (such as Koo antibody). For example, for the 3.0 ml washed cell suspension prepared in Section III step 3 add 90 μl of primary sexing antibody.
2. Incubate the antibody reaction at room temperature for 30 minutes on a rotator at 4 RPM (Cole Parmer Roto-Torque Model 7637 or equivalent).
3. Following the incubation wash the sample by centrifuging at 900×g for 10 minutes to pellet the Sperm cells. Using a pipette, aspirate the supernatant from the sample. Remove as much of the supernatant as possible without disturbing the pellet. Semen pellets can be very soft and as much as 1.0 ml of liquid may remain in the tube following aspiration.
4. Re-suspend the cell pellet to 3.0 mls of PBS using the calibration markings on the side of the centrifuge tube.
   IV. Capture and Separation
1. Transfer 600 μl (1.2 mg) of Bridge Bound Beads (which can be prepared using Procedure 7, described below, using rabbit antimouse IgM bound to Protein A on bead) to a 4 ml cryovial and pull the magnetic beads to the side of the cryovial for 5 minutes in a cryovial magnetic separator. After five minutes in the magnetic separator the supernatant should be clear. Remove the supernatant and re-suspend the beads with 600 μl of PBS. Completely re-suspend the magnetic bead pellet by vortexing vigorously.
2. Add the 600 μl of re-suspended beads directly to the washed antibody labeled cell suspension prepared in section III step 4 and completely mix by gently inverting the tube.
3. Incubate the cell/bead reaction mixture at room temperature for 30 minutes on a rotator at 4 RPM (Cole Parmer Roto-Torque Model 7637 or equivalent).
4. After incubating the cell/bead reaction mixture for 30 minutes transfer the cell/bead suspension to a 4 ml cryovial in the cryovial separator and pull the beads to the side of the cryovial for 10 minutes.
5. After pulling the bead bound cells to the side of the separation chamber for ten minutes carefully aspirate the supernatant from the collection tube while the tube remains fixed in the cryovial magnetic separator. Collect the sample until the dark band of beads begins to slide toward the bottom of the 4 ml cryovial. Do not collect any of the bead bound portion of the solution. It is normal to leave approximately 300 to 400 μl of bead/cell solution in the bottom of the cryovial.
   Procedure 7 Bridge Binding to Beads
1. Place a 10 ml tube of Protein A magnetic Beads (obtained from VICAM, Watertown, Mass.) into the 15 ml magnetic separator for 5 minutes to pull the beads to the walls of the tube.
2. While the tube remains in the magnetic field aspirate the clear supernatant from the tube.
3. Remove the tube containing the magnetic beads from the 15 ml magnetic separator and completely re-suspend the beads in 10 ml of PBS.
4. Repeat steps 1 and 2 a second time for a total of two magnetic washes.
5. Re-suspend the beads in 10 ml of PBS after the second magnetic pull. Completely disperse the beads from the sides of the tube by vortexing vigorously. The Bead concentration is 1.0 mg/ml.
2. Add 5.0 mg (1.0 ml) of bridge antibody to the washed beads and mix by gently inverting the tube.
3. Incubate the binding reaction at room temperature for 2 hours on a rotator at 4 RPM (Cole Parmer Roto-Torque Model 7637 or equivalent).
4. Following the 2 hour incubation remove the tube from the rotator, remove the cap and place in the 15 ml magnetic separator for five minutes to pull the beads to the sides of the tube.
5. While the tube remains in the magnetic field aspirate the clear supernatant from the tube.
6. Remove the tube containing the magnetic beads from the 15 ml separator and completely re-suspend the beads in 10 ml of PBS.
7. Repeat steps 4, 5 and 6 four more times for a total of five magnetic washes.
8. Following the fifth magnetic wash remove the tube from the magnetic separator and re-suspend the beads in 10 ml of PBS. Completely disperse the beads from the sides of the tube by vortexing vigorously. The Bead concentration is now 1.0 mg/ml.
9. Store the beads at 4° C. Beads are stable for at least 1 week.
   Media Used In Procedures and Examples

I. Phosphate Buffered Saline (PBS) Medium, pH 7.5

| | | |
|---|---|---|
| Sodium Phosphate Dibasic Heptahydrate | 2.68 | g |
| NaCl | 8.77 | g |
| Distilled water | 1000 | ml (final volume) |

Adjust the pH to 7.5±0.05 by the drop wise addition of Dilute HCl or NaOH. Filter through 0.22 μm filter prior to use.

II. Basic Media for Bovine Oocyte Maturation and Fertilization In Vitro

| | | |
|---|---|---|
| TL-Hepea (TLH) Medium | | |
| NaCl | 1000 | ml |
| KCl | 6.6600 | g |
| NaHC0₃ | 0.2400 | g |
| NaH₂P0₄ | 0.1680 | g |
| CaCl₂2H₂O | 0.0408 | g |
| MgCl₂6H₂O | 0.3000 | g |
| Hepes | 0.1000 | g |
| Phenol red | 2.4000 | g |
| Na-Lactate (60% syrup) | 0.0100 | g |
| pH: 7.2-7.4 (Regulated with 1 N NaOH) | 1460 | μl |
| Osmotic pressure: 255-265 mOSM | | |
| TL-Stock Medium | | |
| NaCI | 1000 | ml |
| KCI | 6.6600 | g |
| NaHC0₃ | 0.2350 | g |
| NaH₂P0₄ | 2.1040 | g |
| CaCl₂2H₂O | 0.0410 | g |
| MgCl₂6H₂O | 0.3000 | g |
| Hepes | 0.1000 | g |
| Phenol red | 2.4000 | g |
| Na-Lactate (60% syrup) | 0.0100 | g |
| pH is adjusted by C0₂ In the Incubator. | 1460 | μl |
| Osmotic pressure: 280-300 mOSM | | |
| TL-Sperm Stock Medium | | |
| NaCI | 1000 | ml |
| KCI | 5.8200 | g |
| NaHCO₃ | 0.2300 | g |
| NaH₂PO₄ | 2.0900 | g |
| CaCl₂2H₂O | 0.0350 | g |
| MgCl₂6H₂O | 0.2900 | g |
| Hepes | 0.3100 | g |
| Phenol red | 2.3800 | g |
| Na-Lactate (60% syrup) | 0.0100 | g |
| pH: 7.2-7.4 (Regulated with 1 N NaOH) | 3153 | μl |
| Osmotic pressure: 280-300 mOSM | | |

III. Media for IVM (In Vitro Maturation)

| Maturation Medium | | |
|---|---|---|
| | 10 | ml |
| TCM-199 Earle's salt | 8.7 | ml |
| 10% fetal calf serum (FCS) | 1.0 | ml |
| FSH stock | 100 | μl |
| LH stock | 100 | μl |
| or HMG (instead of FSH & LH) | 100 | μl |
| Na-pyruvate stock | 10 | μl |
| Gentamycin stock | 10 | μl |
| Estradiol-17β | | |
| Oocyte Washing Medium For Maturation | | |
| | 30 | ml |
| TLH medium | 27 | ml |
| FCS | 3 | ml |
| Na-pyruvate stock | 300 | μl |
| Gentamycin stock | 30 | μl |

IV. Media for IVF

| Swim Up Medium | | |
|---|---|---|
| | 30 | ml |
| TL-sperm medium | 30 | ml |
| BSA (Fraction V) | 180 | mg |
| Na-pyruvate stock | 100 | μl |
| Gentamycin stock | 30 | μl |
| Fertilization Medium | | |
| | 10 | ml |
| TL-stock medium | 10 | ml |
| BSA (Fraction V) | 60 | mg |
| Na-pyruvate stock | 100 | μl |
| Gentamycin stock | 10 | μl |
| Heparin stock | 20 | μl |
| Oocyte Washing Medium For Fertilization | | |
| | 30 | ml |
| TLH medium | 30 | ml |
| BSA (Fraction V) | 90 | mg |
| Na-pyruvate | 300 | μl |
| Gentamycin stock | 30 | μl |

V. Media For IVC

Basic Medium: BECM (Bovine Embryo Culture Medium)

| | | | 1000 | ml |
|---|---|---|---|---|
| 89 | mM NaCl | | 5.2000 | g |
| 3.2 | mM | KCI | 0.2386 | g |
| 2.0 | mM | CaCI₂2H₂O | 0.2940 | g |
| 0.5 | mm | MgCl₂6HVO | 0.1016 | g |
| 25 | mM NaHCO₃ | | 2,1000 | g |
| 0.35 | mM | NaH2P04 | 0.0483 | g |
| 1.0 | mm | Glutamine | 0.1460 | g |
| 0.5 | mm | Na-pyruvate | 0.0550 | g |
| 10 | mm | Na-Lactate | 1437.18 | μl |
| | | BME amino | 1% | |
| | | acid (100×) | | |
| | | MEM amino | 1% | |
| | | acid (100×) | | |
| | | Phenol red | 1000 | μl |
| | | solution | | |

pH: 7.2-7.4 (Regulated with 1 N NaOH)

Developmental Medium I (for 5 days after insemination)

| | | |
|---|---|---|
| | 10 | ml |
| BECM | 10 | ml |
| BSA (Fatty acid free) | 30 | mg |
| Gentamycin | 10 | μl |

Developmental Medium II (from 5 days after insemination)

| | | |
|---|---|---|
| | 10 | ml |
| BECM | 9 | ml |
| 10% Fetal calf serum (FCS) | 1 | ml |
| Gentamycin | 10 | μl |

### EXAMPLE 1

### ICC Separation Time Courses with Bull Semen

Four time studies (Samples 1-4) were conducted as described below.

Sample 1 was cooled to Room Temperature, i.e., actually 28° C., on the bench. Samples 2-4 were cooled to 4° C., 12° C. and 16° C., respectively, using a circulating water bath.

1.0 ml separations using S-Beads (VICAM, Watertown, Mass.) were performed at t=0, 2, 4, 6, 8, 12 and 24 hours. Samples of the washed cells at each time point were labeled with Koo Ab for immediate ICC analysis. Frozen control and final sexed samples were kept for FISH analysis. For each temperature point an ejaculate was collected into a 15 ml conical centrifuge tube. The tube was immediately transferred to a 250 ml beaker containing 200 ml of water at 32° C. This beaker was immediately placed into a water bath at the desired temperature and allowed to cool and sampled over a 24 hour period and separation was performed as described in Procedure 5, above. A 1.0 ml sample was taken at t=0, 2, 4, 6, 8, 10, 12 and 24 hours post ejaculation and separated as described in the 1.0 ml separation protocol (Procedure 5, above).

ICC labeling of the samples (one sample at each time point) was performed on 100 μl of the 2× washed semen sample, which was added to 150 μl of PBS (10 mM sodium phosphate and 150 mM NaCl) and labeled with 6 μl of Koo antibody (1.0 mg/ml Koo in PBS). The sample was incubated at room temperature (28° C.) for 30 minutes with occasional mixing. Following the incubation the sample was diluted by the addition of 1.0 ml of PBS and pelleted for ten minutes at 900×g at room temperature. The washed pellet was resuspended to 250 μl with PBS and 12 μl of a fluorescein labeled polyclonal Rabbit anti mouse IgM was added. This reaction was incubated for 30 minutes at room temperature in the dark. Following the incubation the sample was diluted by addition of 1.0 ml of PBS and immediately evaluated for % ICC positive cells using a fluorescent Microscope and the appropriate excitation and emission filters.

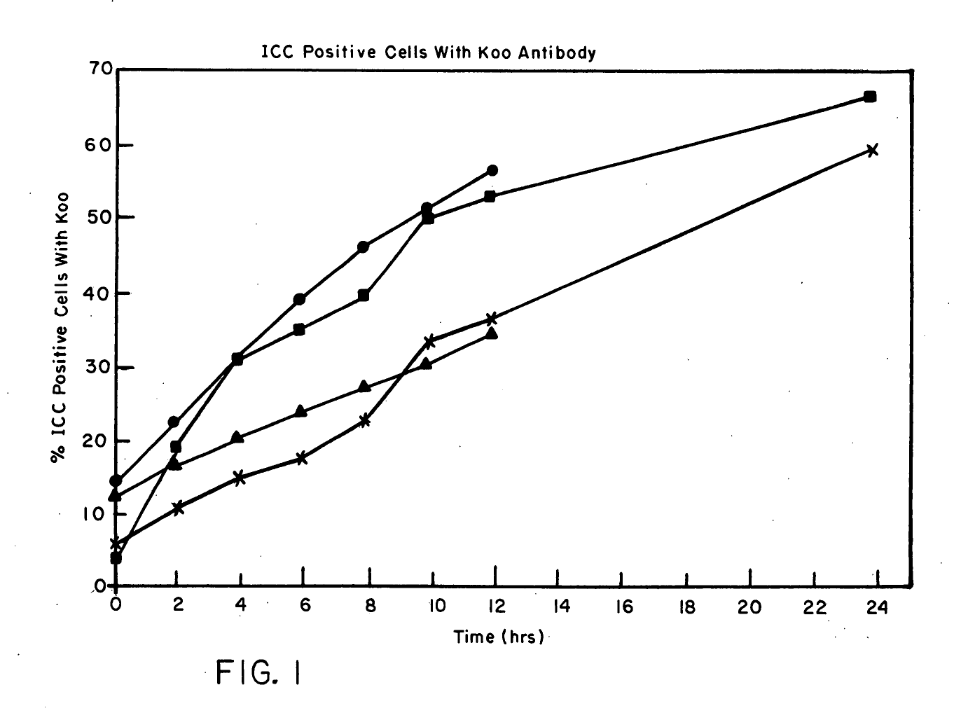

The results of these experiments are shown in Table 1, which shows ICC positive cells at each time point for four temperatures. Data for each temperature set was obtained using a single ejaculate. FIG. 1 illustrates the increase in the percent of sperm cells ICC positive as a function of time at different temperatures.

TABLE 1
Percent Of Cells ICC Positive With Koo Antibody Over Time

| Time (hours) | 4° C. | 12° C. | 16° C. | 28° C. |
|---|---|---|---|---|
| 0 | 14.1 | 3.8 | 12.3 | 5.6 |
| 2 | 22.4 | 18.9 | 16.9 | 10.8 |
| 4 | 31.0 | 30.5 | 20.5 | 15.1 |
| 6 | 39.2 | 34.9 | 23.9 | 17.6 |
| 8 | 46.1 | 39.5 | 27.1 | 23.7 |
| 10 | 51.1 | 49.8 | 30.4 | 33.2 |
| 12 | 56.3 | 52.8 | 34.3 | 36.2 |
| 24 | — | 66.3 | — | 59.2 |

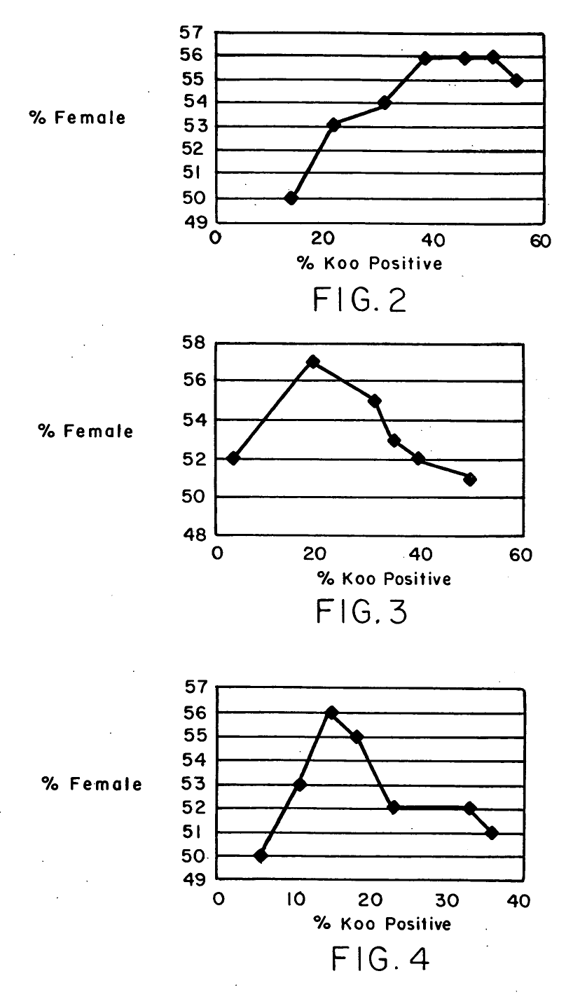

Each of the four temperatures shows an increase in the total percentage of ICC positive cells as incubation time increases. The data is plotted in FIG. 2. It appears that there is little difference between the curves at 4° C. (●) and at 12° C. (▪) or between the curves at 16° C. (▴) and 28° C. (x). FISH data, as shown in Table 2, is used to indicate where on these curves the sexing window appears at these different temperature conditions. For the FISH procedure, probes were obtained from Cambio Ltd. of the United Kingdom and their recommended protocol was followed.

TABLE 2
Percent Female Cells In Samples Separated Over Time

| Time (hours) | 4° C. | 12° C. | 16° C. | 28° C. |
|---|---|---|---|---|
| Control | 48.3 | 48.5 | 50.4 | 50.5 |
| 0 | 49.6 | 52.1 | 49.2 | 49.8 |
| 2 | 52.9 | 57.2 | 51.5 | 53.2 |
| 4 | 54.1 | 54.9 | 53.8 | 56.4 |
| 6 | 55.9 | 52.5 | 50.8 | 55.1 |
| 8 | 56.1 | 52.4 | 54.7 | 51.5 |
| 10 | 56.2 | 50.6 | 51.0 | 52.1 |
| 12 | 54.6 | 47.1 | 54.4 | 50.7 |
| 24 | — | — | — | — |

It should be noted that, during the course of these experiments, the actual centrifugation, washing, labeling and separations steps were completed on the bench at ambient temperature, which was 28° C. in this case. That is, ambient is the temperature for processing once the raw ejaculate has been incubated at the indicated temperature for the indicated time.

FIGS. 2-4 are graphs of the data in Table 1 plotted against the data in Table 2. These graphs illustrate the opening and closing of the window for sexing, i.e., treating cells to separate preferentially male from female cells to provide a female bias in calving.

Two non refrigerated centrifuges were used for these experiments.

This experiment was repeated partially, thereby obtaining the data shown in Tables 3 and 4 below. In the repeated experiment, the ambient temperature was 21° C.

TABLE 3
Percent of Cells ICC Positive

| Time | 4° C. Incubation | 12° C. Incubation | Room Temp (21° C.) |
|---|---|---|---|
| 0 | 17.6 | 14.2 | 14.8 |
| 2 | 20.8 | 21.4 | 16 |
| 4 | 33.6 | 27.5 | 11 |
| 6 | 34.2 | 15.4 | 19.8 |
| 8 | 35.9 | 32 | 20 |
| 10 | 36.2 | 42.4 | 15.7 |
| 12 | 35.6 | 32.8 | 15.6 |

TABLE 4
Percent of Female Cells

| Time | 4° C. Incubation | 12° C. Incubation | Room Temp (21° C.) |
|---|---|---|---|
| Control | 49.4 | 50.2 | Not done |
| 0 | 50.4 | 50.7 | ″ |
| 2 | 54.6 | 53.5 | ″ |
| 4 | 56.8 | 54.5 | ″ |
| 6 | 57.6 | 59.3 | ″ |
| 8 | 57.4 | 57.1 | ″ |
| 10 | 56.4 | 52.9 | ″ |
| 12 | 54.1 | 51.1 | ″ |

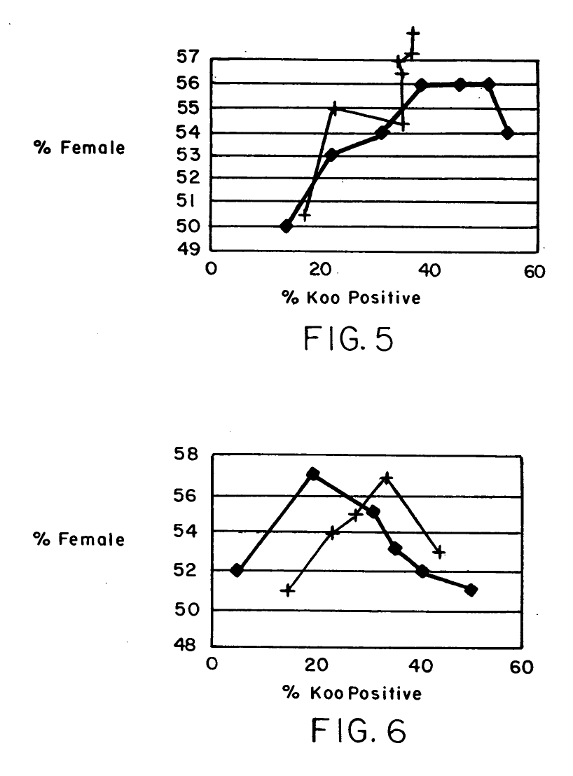

FIGS. 5 and 6 show graphs comparing data collected when room temperature was 28° C. (♦) and 21° C. (+).

### EXAMPLE 2

### Reproducibility of Sexing Window

10 ejaculates were processed by the Semen Sexing protocol (Protocol 5), as described above (each ejaculate was split into 1.0 ml aliquots and the sexed samples from a given temperature were pooled after the magnetic separation step). Following completion of the separation process each ejaculate was extended in egg yolk citrate extender and frozen using industry standard methods. FISH analysis was performed to determine the female bias achieved in each sample. Table 5 shows data from quality assessment of the frozen semen samples. Semen which had been stored under liquid nitrogen for 4 days was thawed in a warm water bath and semen quality evaluated immediately for % motility and for forward motility (forward motility is described as 1=poor to 5=excellent). Semen, then, was incubated on a warming table for 4 hours and re-evaluated. Control samples were removed, extended and frozen at this point after the samples were incubated for 6 hours at 12° C. Sexed samples were the remainder of the ejaculate that was processed as described. Post-thaw motility is scored as follows: for a score such as 70-4 seen with the bull Cool Cat on the first line of the Table 5, 70 is the percent of sperm that are motile post-thaw and 4 is the grade of motility post-thaw, on a scale of 1 (poor) to 5 (excellent).

TABLE 5

| Bull | Process | Sexed or | Ejaculate | Post Thaw Motility | |
|---|---|---|---|---|---|
| Name | Temp. | Control | Motility | Immediate | 4 hour |
| Cool | NA | Control | 90% | 70-4 | ND |
| Cat | | | | | |
| Cool | RT | Sexed | 90% | 55-3 | 55-4 |
| Cat | | | | | |
| Disco | NA | Control | 85% | 70-4 | 60-4 |
| Disco | RT | Sexed | 85% | 55-4 | 50-4 |
| Leader | NA | Control | 85% | 45-4 | 5-1 |
| Leader | RT | Sexed | 85% | 20-2 | 5-2 |
| Disco | NA | Control | 85% | 70-4 | 40-4 |
| Disco | RT | Sexed | 85% | 55-4 | 30-3.5 |
| Lance | NA | Control | 85% | 75-4 | 60-4 |
| Lance | RT | Sexed | 85% | 60-4 | 55-4 |
| Cool | NA | Control | 0.85 | 65-4 | 65-4 |
| Cat | | | | | |
| Cool | 12° C. | Sexed | 0.85 | 30-3+ | 30-3+ |
| Cat | | | | | |
| Disco | NA | Control | 85% | 65-4 | 70-4 |
| Disco | 12° C. | Sexed | 85% | 75-4 | 75-4 |
| | | 12° C. | | | |
| Disco | RT | Sexed RT | 85% | 55-4 | 55-4 |
| Limpid | NA | Control | 85% | 65-4 | 55-4 |
| Limpid | 12° C. | Sexed | 85% | 60-4 | 50-4 |
| Cool | NA | Control | 80% | 60-4 | 60-4 |
| Cat | | | | | |
| Cool | 12° C. | Sexed | 80% | 30-3+ | 40-4 |
| Cat | | | | | |
| Disco | NA | Control | 80% | 65-4 | 70-4 |
| Disco | 12° C. | Sexed | 80% | 70-4 | 70-4 |
| Limpid | NA | Control | 80% | 50-3+ | 50-3+ |
| Limpid | 12° C. | Sexed | 80% | 45-3+ | 45-3+ |
| Midas | NA | Control | 80% | 70-4 | 70-4 |
| Midas | 12° C. | S-bead | 80% | 60-4 | 65-4 |
| | | Sexed | | | |
| Midas | 12° C. | Koo Sexed | 80% | 60-4 | 65-4 |

NA Not Applicable
ND Not Determined

### EXAMPLE 3

### Comparison of Sexing Between S-Bead and Antibody Chase Capture Methods

Following 6 Hour, 12° C. Incubation

A single ejaculate was collected from a bull using an artificial vagina and the sample was immediately cooled to 12° C. (as described in Procedure 5) and kept at this temperature for 6 hours. Following the 6 hour, 12° C. incubation the raw ejaculate was split into three fractions and treated as follows.

Fraction 1 was immediately extended and frozen without further processing as control.

Fraction 2 was sexed using the Koo antibody in a chase capture method as described in Procedure 6, above.

Fraction 3 was sexed using the S-Bead method as described in Procedure 5, above.

The results of FISH analysis of the control and sexed fractions are given in Table 6. The control fraction shows the expected 50% female to male ratio while both the Koo antibody selected and S-Bead selected population exhibit an enhanced female cell bias of 56.2% in a genetic assay, a bias that would be predicted to be higher in sexed samples by roughly 5% if a biological assay were used to evaluate (for example, IVF), in which case the control would be about 50% and the sexed samples would be about 61% female.

TABLE 6

| Sample | Female Bias (FISH) |
|---|---|
| Midas Raw Ejaculate (control) | 50.1% |
| Sexed By Chase Capture (Fraction 2) | 56.2% |
| Sexed By S-Bead (Fraction 3) | 56.2% |

### EXAMPLE 4

### Cows Inseminated with Treated Semen Conceive

Semen samples (ejaculates) from bulls in a sire lineup were collected and the semen was treated with Koo antibody and beads using the procedures in accord with the present invention, as described above. The treated semen was used to inseminate cows that had been superovulated. Embryos were flushed from cows and sexed using methods based on polymerase chain reaction with sex-specific primers, as described above, or according to a commercial method employed by a commercial laboratory (EmTran) that carried out some of the embryo sexing. The sex of the embryos from the super-ovulated cows inseminated with treated semen is shown in Table 7.

TABLE 7

| | | | | | % Female |
|---|---|---|---|---|---|
| Trial | # Cows | # Embryos | # Female | # Male | Bias |
| 1 | 5 | 36 | 23 | 13 | 64% |
| 2 | 8 | 54 | 31 | 23 | 57% |
| Total | 13 | 90 | 54 | 36 | 60% |

One trial was aborted when fertility was so poor that the trial could not be continued (data not shown).

### EXAMPLE 5

### Scottish Bulls

Semen samples (ejaculates) from bulls from a sire lineup in Scotland were collected and the semen was treated with Koo antibody and beads using the procedures in accord with the present invention, as described above. Samples of treated semen were analysed by fluoresence in situ hybridization (FISH) using different color labels for X and Y probes, in order to determine the genetic percentages of X- and Y-bearing sperm in samples from each bull taken before and after treatment. The cell count and sex ratios of sperm, as determined by FISH analysis with dual-color probes, in treated and untreated samples from split ejaculates of the bulls, is shown in Table 8.

TABLE 8

| | | Number of | Number of | |
|---|---|---|---|---|
| | | X-bearing | Y-bearing | |
| | | Sperm | Sperm | % Female |
| Bull | Process | Counted | Counted | Bias |
| Panama | Control | 257 | 278 | 48% |
| | Sexed | 372 | 283 | 57% |
| Bonus | Control | 253 | 294 | 46% |
| | Sexed | 311 | 238 | 56% |
| Roberto | Control | 254 | 271 | 48% |
| | Sexed | 355 | 286 | 55% |
| Leslie #1 | Control | 265 | 302 | 47% |
| | Sexed | 303 | 265 | 63% |
| Leslie #2 | Control | 239 | 240 | 50% |
| | Sexed | 332 | 246 | 57% |
| Back up | Control | 332 | 336 | 50% |
| | Sexed | 285 | 108 | 72% |
| Roberto | Control | 291 | 254 | 53% |
| | Sexed | 380 | 338 | 53% |

### EXAMPLE 6

### Irish Bulls

Semen from Bulls in a sire lineup was treated and used to inseminate both cows and heifers on working dairy farms. The first repeat rate (need for re-insemination due to lack of conception) was 24% for control cows (55 cows total) and 26% for cows receiving treated semen (66 cows total), showing good fertility in cows, where conception is more difficult, as well as in heifers; an unusual result for a sexed semen trial where historically conception using other methods of sexing is so poor as to be unworkable on farm. The sex of calves on the ground from this trial with treated semen is shown in Table 9. The increase in percent female births is +16%, which would correspond, if female births had been 50% in the control, to a female bias of 66%, or basically 2 heifers to 1 bull. The average of female births is lower that 50%, it is 42%, with biased samples giving 57% female with calves of normal morphology and no birth defects observed.

TABLE 9

| Farmer | Bull Calves | Heifer Calves |
|---|---|---|
| BF | 3 | 4 |
| CG | 7 | 13 |
| HP | 16 | 18 |
| JD | 5 | 3 |
| MR | 8 | 13 |
| NW | 43 | 59 |
| Totals | 82 | 110 |
| Previous Female Bias 42%* | | |
| Female Bias this Trial 57% | | |

*A prior survey, reportedly by the Irish govermental agency Taegac, of 2,369 cows in a 3 year study in Ireland had shown a female bias of 42% with unsexed semen.

### EXAMPLE 7

### Sex Bias of Embryos Generated During In Vitro Fertilization

Semen was collected from the indicated bulls. All bulls but Katahdin were from the sire line-up of an AI company producing semen from genetically superior proofed bulls to be used for artificial insemination of cows and heifers in working dairy herds. Semen from all bulls except Katahdin was collected and fractionated at the AI company, where fractionation was carried out less than one hour post-collection and at ambient temperature. Semen from Katahdin could not be fractionated on farm, so the collection was shipped cold and fractionated about six hours post-collection (bringing this semen into an optimal time/temperature combination in the sexing window). The fractionation was performed using the protocol of Procedure 6, above. Control semen was prepared as described in Procedure 5, section I, above. Semen was frozen and packed into straws for use during in vitro fertilization. The fertilization was carried out as described in Procedures 2 and 3, above. Embryos were obtained. A portion of each embryo was subjected to PCR analysis for sex determination as described in Procedure 4, above. The sex bias of embryos generated by in vitro fertilization with sexed and control semen is shown in Table 10.

TABLE 10

| | Male | Female | Total | % Female |
|---|---|---|---|---|
| Semen Source | Embryos | Embryos | Embryos | Bias |
| All Control Semen | 62 | 73 | 135 | 54.1% |
| Embryos | | | | |
| All Sexed Semen | 154 | 250 | 404 | 62% |
| Embryos | | | | |
| Bull Katahdin | 26 | 69 | 95 | 72.6% |
| Bull Ayershire 74 | 32 | 40 | 72 | 55.6% |
| Bull Jersey 424 | 47 | 60 | 107 | 56.1% |
| Bull Jersey 329 | 49 | 81 | 130 | 62.3% |

### EXAMPLE 8

### Reactivity of Human Sperm with Koo Antibody

Human sperm was collected and subjected to immunocytochemistry, as described above. Human sperm was from donors negative for human immunodeficiency virus and hepatitis B virus by standard clinical screens. Semen was processed at ambient temperature within several hours of collection. In this experiment, a sperm suspension was prepared by diluting 200 μl raw ejaculate in 1 mL PBS—giving a cell count of 4×10(6)/mL. 250 μl of the suspension was placed in a 10.5 ml Eppendorf centrifuge tube and mixed with 25 μl of either antibody from hybridoma HB9070 or HB9071, both from ascites (antibody concentration ca. 2 mg/ml as ascites that was produced at a commercial laboratory by standard methods for obtaining ascites). The mixture was incubated for 30 minutes at room temperature. It was centrifuged at low speed (about 900×g) for 10 minutes and the supernatant was discarded. 300 μl of phosphate buffered saline (standard formulation except thimerosal was omitted) was added and 20 μL of FITC-conjugated second antibody (goat anti-mouse IgM, sourced from ICN/Cappel, Aurora, Ohio) was added and incubated 20 minutes at room temperature in the dark before centrifugation for 10 minutes at 900×g. The supernatant was discarded and 300 μl PBS was added to wash the cells. After another centrifugation for 10 minutes at 900×g, supernatant was discarded and cells were scored for fluoresence using a microscope, where Koo positivity over time is measured as a function of temperature to which sperm are exposed immediately post-collection. The FITC filter is used for this work. The reactivity of human sperm with Koo antibodies is shown in Table 11, confirming that, as we found with other species, a subpopulation of human sperm can react with the Koo antibody.

TABLE 11

| Koo Antibody Used | % Human Sperm Antibody Positive |
|---|---|
| HB9070 | 50% (41 cells out of 82 total) |
| HB9071* | — |

*no signal visible due to poor experiment

### EXAMPLE 9

### Reactivity of Pig Sperm with Koo Antibody

Pig sperm from a Yorkshire White named Aftershock was collected and shipped as raw semen in a cooler to the laboratory, where it was processed. Processing varied from within hours of collection to up to 24 hours post-collection in various experiments. The ejaculate used for this experiment consisted of 50 ml containing 160×10(6) cells/ml. For ICC, 10×10(6) cells in 1 ml BTS buffer (“Beltsville Thawing Solution,” sold by Minitube of America) were mixed with Koo HB9071 antibody, which had been stored at 13.5 ug/ml but was diluted for this experiment by taking 10 μl antibody into 139 μl and using 2511 of this antibody dilution to mix with sperm. This mixture was stored overnight at 4° C. The next day, sperm were washed by centrifuging at 1500 rpm for 5 min and 400 μl BTS was added containing 10 μl FITC-rabbit anti-mouse IgM (Pierce) reconstituted according to the manufacturer's instructions. Cells were examined under the microscope. The reactivity of pig sperm to Koo antibodies is shown in Table 12.

TABLE 12

| Antibodies Used | % Pig Sperm Antibody Positive |
|---|---|
| HB9071 plus secondary Ab | 34% |
| reconstituted 3 weeks | |
| prior to experiment | |
| HB9071 plus secondary Ab | 28% |
| reconstituted on same | |
| day as experiment | |

### EXAMPLE 10

### Reactivity of Mouse Sperm with Koo Antibody

As for human and pig sperm, with mouse sperm Koo monoclonal antibodies recognize only a subpopulation of these cells, an attribute required of a sexing reagent. Immunocytochemistry was performed in solution according to the procedure of Ali and coworkers (Archives of Andrology, 24:235-45 (1990)) on washed mouse sperm that had been collected from the vas deferens and epididymis of Balb/C mice. The results in Table 13 are reported as averages of two independent experiments and show the Koo positivity of mouse sperm.

TABLE 13

| Antibody Used | % Mouse Sperm Antibody Positive |
|---|---|
| HB9070 | 52 |
| HB9071 | 54 |

Koo-reactive mouse sperm can be cleared from solution by mixing sperm with magnetic beads coated with Koo antibody and capturing the beads with a magnet. Sperm suspensions of about one million sperm in a volume of 300 μl were exposed to either no beads, Koo beads or anti-Salmonella beads (VICAM, Watertown, Mass.) for 30 minutes with gentle rotation at about 2 rpm on a small (diameter ca. 20 cm) rotator. Beads were added at an approximate ratio of 5 beads to one sperm. Magnetic capture was carried out for 15 minutes, by open-field capture using a rare earth magnet (VICAM, Watertown, Mass.). Immunocytochemistry on the sperm remaining in solution was carried out. The results in Table 14 are presented as an average of two independent experiments for each experimental condition and show that Koo positive mouse sperm can be separated from the supernatant.

TABLE 14

| | % Koo-reactive Mouse Sperm in |
|---|---|
| Bead Treatment | Supernatant |
| None | 54% |
| Salmonella Beads | 48% |
| Koo HB9070 Beads | 12% |

### EXAMPLE 11

### Sex of Mouse IVF Embryos Produced by Treated Sperm

In vitro fertilization of mouse eggs with treated sperm produces predominantly female embryos. Mouse sperm were isolated and treated with sexing beads by procedures in accord with the present invention, as described above. Sperm remaining in suspension were evaluated for reactivity to Koo antibody and were used to fertilize eggs that were cultured to the blastocyst stage. Colchicine was added and chromosome squashes were prepared and karyotyped. The ratio of Y-chromosomes to total chromosomes directly reflects the sex ratio of the embryonic cells, provided one assumes that male and female embryos have similar rates of cell division from fertilization to the blastocyst stage (Experiment 1). If male cells divide faster, as has been proposed by some scientists, this assay will produce conservative results and underreport female bias. Alternatively (Experiments 2-4), individual eggs in separate containers were fertilized, grown to the blastocyst stage, and made into chromosome squash preps for karyotyping. This assay should not be affected by different rates of cell division if they exist between male and female cells, but the number of embryos that can be scored is extremely limited. Table 15 reports the sex of mouse IVF embryos produced by treated sperm.

TABLE 15

| | Antibody Reactivity of Sperm | | Embryo Sex | | |
|---|---|---|---|---|---|
| Experiment | % Ab⁺ | % Ab⁻ | Male | Female | % Female |
| 1*pooled | 25 | 75 | 10 | 44 | 81% |
| embryos | | | | | |
| 2 individual | 11 | 89 | 0 | 3 | 100% |
| embryos | | | | | |
| 3 individual | 19 | 81 | 0 | 7 | 100% |
| embryos | | | | | |
| 4 individual | 19 | 81 | 0 | 4 | 100% |
| embryos | | | | | |

*By chromosome ratios. All others by individual embryos.

### EXAMPLE 12

### Separation of Spermatozoa Using a Magnetic Separator

Magnetic beads made by a co-precipitation process and coated with protein A were used. The beads were bound to an excess of rabbit anti-mouse IgM antibody for 2 hours, washed magnetically 5× and resuspended into phosphate buffered saline (PBS). Magnetic washing was performed by placing the suspension of magnetic beads into a dipole magnetic separator for 5 minutes to pull the beads to the walls of the tube, aspirating the clear supernatant from the tube, and resuspending the magnetic beads in 10 ml of PBS.

An aliquot (790 μl) of a freshly collected bull ejaculate was diluted to 8.0 ml with PBS and then washed 2 times by pelleting by centrifugation at 900×g for ten minutes and resuspension in PBS.

Washed cells were re-suspended in 6.0 ml of PBS and 360 μg of a primary sexing antibody was added (Koo et al., Hum. Genet. 58(1):18-20, 1981; U.S. Pat. No. 4,680,258 to Hammerling et al.). The sample was allowed to bind for thirty minutes at room temperature with gentle mixing. Bound cells were washed 1× by pelleting by centrifugation at 900×g for ten minutes.

Washed cells were re-suspended in 6.0 ml of PBS and 1.2 ml of rabbit anti-mouse IgM-bound magnetic beads were added to bring the solution to 0.2 mg beads/ml solution. Sample was allowed to bind for thirty minutes at room temperature with gentle mixing.

The sample then was placed in a magnetic separator and the beads were captured in the magnetic field for ten minutes. Female cells (X chromosome bearing spermatozoa) were retrieved from the bottom of the device by opening the stopcock and draining in a controlled flow.

Cells were then characterized by measuring cell count and motility of the cells eluted from the magentic separator device. A summary of the cell numbers and motility before and after processing with the magnetic separator device is provided in Table 16 below.

TABLE 16

| Property | Value |
|---|---|
| Volume of semen sample | 0.79 ml |
| Cell concentration in ejaculate | 1253 × 10⁶ |
| Total cells into separation | 990 × 10⁶ |
| Motility of cells immediately post ejaculation | 75% |
| Cell concentration into separator device | 116 × 10⁶ |
| Cell concentration of the eluate from separator | 77 × 10⁶ |
| Input cells recovered from separator | 66.4% |
| Motility of the recovered spermatozoa | 75% |

Collected cells were then extended using an egg yolk extender. Commercially available extenders that can be used include, for example, Biladyl®, Triladyl® and Biociphos Plus™. The cells then were transferred to straws and frozen using standard freezing techniques.

Frozen straws were thawed approximately 2 months post-freezing and the semen used to produce 109 embryos via in vitro fertilization.

The sex of each individual embryo was determined by sexing through PCR amplification of the ZFX and ZFY regions of the X and Y chromosomes, respectively, as described above. Also, see Kirkpatrick and Monson, J. Reprod. Fertil. 98:335-340 (1993).

All embryos (from 8-cell to hatched blastocyst stage) were produced by in vitro fertilization (IVF). A few embryonic cells were extracted from these embryos and put into a 250 μl PCR tube containing 5 μl lysis buffer (2% 2-mercaptoethanol, 0.01% SDS, 10 mM EDTA, 10 mM Tris pH 8.3, proteinase K, 222 μg/ml). All embryonic cells were lysed at 55° C. for 2 hr, and proteinase K was inactivated at 98° C. for 10 min. Then, the sample was ready for PCR embryo sexing, as described above.

The results of the PCR sex determination of the IVF embryos indicated that there were 85 female embryos and 24 male embryos, for an apparent sex bias of 78% in favor of females.

The invention has been described in detail including preferred embodiments thereof. However, modifications and improvements within the scope of this invention will occur to those skilled in the art. The above description is intended to be exemplary only. The scope of this invention is defined only by the following claims and their equivalents.

All patent and literature references disclosed hereinabove hereby are incorporated by reference in their entirety.

1. A method for treating a specimen of semen containing sperm cells to increase the relative number of sperm cells of a selected sex type in a treated specimen to increase the potential for conceiving an offspring of the selected sex, the method comprising holding the specimen at a predetermined temperature for at least a predetermined time and, thereafter, treating the specimen of semen to increase the relative ability of a at least a portion of the semen to conceive an offspring of the desired sex.

2. The method of claim 1, wherein the treating step comprises contacting the sperm cells with an agent that preferentially effects sperm cells of a selected sex type.

3. A method for treating a specimen of semen containing sperm cells to increase the relative number of sperm cells of a selected sex type in a treated specimen to increase the potential for conceiving an offspring of the selected sex, the method comprising separating the semen into two components comprising a first component having a higher number of sperm of the selected sex type than sperm of a non selected sex type and a second component having a higher number of sperm of the non selected sex type relative to sperm of the selected sex type, wherein the separating step is performed after a predetermined percent of the sperm cells exhibit a punctate pattern, which is capable of determination by labeling the sperm cells with Koo antibody and determining the percent of cells labeled.

4. The method of claim 3, wherein the separating step comprises contacting the sperm with a cell binding agent; permitting the sperm of the non selected sex type to preferentially bind to the cell binding agent, and separating the cell binding agent with preferentially bound sperm of the non selected sex type from non bound sperm, thereby providing said first component.

5. The method of claim 3, wherein the first component comprises a higher number of X-chromosome bearing sperm relative to Y-chromosome bearing sperm and the second component comprises a higher number of Y-chromosome bearing sperm relative to X-chromosome bearing sperm.

6. The method of claim 3, wherein the separating step is performed after 20% of the sperm cells exhibit the punctate pattern.

7. The method of claim 3, further comprising cooling the semen before performing the separating step.

8. The method of claim 3, further comprising collecting the semen and immediately cooling the semen.

9. The method of claim 8, wherein the semen is cooled to at least about 16° C.

10. The method of claim 8, wherein the semen is cooled to at least about 12° C.

11. A method for increasing the probability of producing an offspring of a mammalian species having a selected sex, said method comprising: separating semen containing sperm cells of the mammalian species into two components comprising a first component having a higher number of sperm of the selected sex type than sperm of a non selected sex type and a second component having a higher number of sperm of the non selected sex type relative to sperm of the selected sex type, wherein the separating step is performed after a predetermined percent of the sperm cells exhibit a punctate pattern, which is capable of determination by labeling the sperm cells with Koo antibody, and fertilizing an egg of the mammalian species with the first component.

12. The method of claim 11, wherein the separating step comprises contacting the sperm with a cell binding agent; permitting the sperm of the non selected sex type to preferentially bind to the cell binding agent, and separating the cell binding agent with preferentially bound sperm of the non selected sex type from non bound sperm, thereby providing said first component.

13. The method of claim 11, wherein the first component comprises a higher number of X-chromosome bearing sperm relative to Y-chromosome bearing sperm and the second component comprises a higher number of Y-chromosome bearing sperm relative to X-chromosome bearing sperm.

14. The method of claim 11, wherein the separating step is performed after 20% of the sperm cells exhibit the punctate pattern.

15. The method of claim 11, further comprising cooling the semen before performing the separating step.

16. The method of claim 11, further comprising collecting the semen and immediately cooling the semen.

17. The method of claim 16, wherein the semen is cooled to at least about 16° C.

18. The method of claim 16, wherein the semen is cooled to at least about 12° C.

19. The method of claim 11, wherein the first component contains two-thirds viable X-chromosome bearing sperm and one-third viable Y-chromosome bearing sperm.

20. The method of claim 11, wherein twice as many female calves are born than male calves when the treated semen is used to fertilize a plurality of eggs.

21. A method for separating a selected population of cells from a sample of semen, the method comprising:
cooling the semen to a predetermined temperature;
waiting until a predetermined percent of the cells exhibit a punctate pattern, which is capable of determination by labeling the sperm cells with Koo antibody;
contacting the sample with a cell binding agent to preferentially bind to the selected population of cells for a time sufficient for the cell binding agent to bind the selected cells; and
separating the selected cells and the cell binding agent to provide a treated sample containing non selected cells.

22. The method of claim 21, wherein the treated sample comprises a population of cells wherein a first number of cells exhibiting preferred sex type is at least about 10% greater than a second number of cells exhibiting a non preferred sex type.

23. The method of claim 21, wherein the treated sample comprises a population of cells wherein a first number of cells exhibiting a preferred sex type is at least about 20% greater than a second number of cells exhibiting a non preferred sex type.

24. The method of claim 21, wherein the treated sample comprises a population of cells wherein a first number of cells exhibiting a preferred sex type is at least about 50% greater than a second number of cells exhibiting a non preferred sex type.

25. The method of claim 21, wherein the treated sample comprises a population of cells wherein a first number of cells exhibiting a preferred sex type is at least about 100% greater than a second number of cells exhibiting a non preferred desired sex type.

26. The method of claim 21, wherein the cell binding agent comprises a bead support having a diameter of 0.1 to 2 microns.

27. The method of claim 21, wherein the number of cells in the selected population of cells is greater than about 1×10⁵ cells/ml.

28. A method of insemination, the method comprising obtaining semen containing a population of spermatozoa according to the method of claim 21 and inseminating a mammal with the treated sample containing a population of spermatozoa.

29. The method of claim 28, wherein the mammal is selected from the group consisting of cattle, sheep, pigs, goats, horses, dogs and cats.

30. The method of claim 28, wherein the semen is cooled to at least about 16° C.

31. The method of claim 28, wherein the semen is cooled to at least about 12° C.

32. A method of increasing the percentage of mammalian offspring of a predetermined sex, the method comprising:
cooling a specimen of semen containing a population of cells to a predetermined temperature;
waiting until a predetermined percent of the cells exhibit a punctate pattern, which is capable of determination by labeling the sperm cells with Koo antibody;
contacting the sample with a cell binding agent to preferentially bind to a selected population of cells for a time sufficient for the cell binding agent to bind the selected cells; and
separating the selected cells and the cell binding agent to provide a treated sample containing non selected cells; and
administering at least a portion of the treated sample to the reproductive tract of a female mammal.

33. The method of claim 32, wherein the treated sample comprises a population of cells wherein a first number of cells exhibiting a preferred sex type is at least about 10% greater than a second number of cells exhibiting a non preferred sex type.

34. The method of claim 32, wherein the treated sample comprises a population of cells wherein a first number of cells exhibiting a preferred sex type is at least about 20% greater than a second number of cells exhibiting a non preferred sex type.

35. The method of claim 32, wherein the treated sample comprises a population of cells wherein a first number of cells exhibiting a preferred sex type is at least about 50% greater than a second number of cells exhibiting a non preferred sex type.

36. The method of claim 32, wherein the treated sample comprises a population of cells wherein a first number of cells exhibiting a preferred sex type is at least about 100% greater than a second number of cells exhibiting a non preferred sex type.

37. The method of claim 32, wherein the cell binding agent comprises a bead support having a diameter of 0.1 to 2 microns.

38. The method of claim 32, wherein the number of cells in the selected population of cells is greater than about 1×10⁵ cells/ml.

39. The method of claim 32, wherein the mammal is selected from the group consisting of cattle, sheep, pigs, goats, horses, dogs and cats.

40. The method of claim 32, wherein the semen is cooled to at least about 16° C.

41. The method of claim 32, wherein the semen is cooled to at least about 12° C.

42. A method for fractionating an ejaculate of a mammal, the method comprising:
obtaining an ejaculate;
cooling the ejaculate to a predetermined temperature;
waiting for a period of time between about 2 hours and about 24 hours after collection of the ejaculate; and
fractionating the ejaculate to obtain a selected population of cells.

43. The method of claim 42, wherein the selected population of cells comprises a higher number of X-chromosome bearing sperm relative to Y-chromosome bearing sperm and the second component comprises a higher number of Y-chromosome bearing sperm relative to X-chromosome bearing sperm.

44. The method of claim 42, wherein the fractionating step comprises contacting the ejaculate with a cell binding agent; permitting the sperm of a non selected sex type to preferentially bind to the cell binding agent, and separating the cell binding agent with preferentially bound sperm of the non selected sex type from non bound sperm, thereby providing a selected population of cells.

45. A method of insemination, the method comprising obtaining the selected population of cells according to the method of claim 44 and inseminating a mammal with the selected population of cells.

46. The method of claim 45, wherein the mammal is selected from the group consisting of cattle, sheep, pigs, goats, horses, dogs and cats.

47. The method of claim 45, wherein the semen is cooled to at least about 16° C.

48. The method of claim 45, wherein the semen is cooled to at least about 12° C.

49. The method of claim 42, wherein the fractionating step is carried out between about 2 hours and about 12 hours after collection of the ejaculate.

50. The method of claim 42, wherein the fractionating step is carried out between about 4 hours and about 8 hours after collection of the ejaculate.

51. The method of claim 42, wherein the fractionating step is carried out at about 6 hours after collection of the ejaculate.

52. The method of claim 42, wherein the semen is cooled to at least about 16° C.

53. The method of claim 42, wherein the semen is cooled to at least about 12° C.

54. A method of insemination, the method comprising obtaining semen containing a population of spermatozoa according to the method of claim 42 and inseminating a mammal with the selected population of cells.

55. The method of any of claim 54, wherein the mammal is selected from the group consisting of cattle, sheep, pigs, goats, horses, dogs and cats.

56. The method of claim 54, wherein the semen is cooled to at least about 16° C.

57. The method of claim 54, wherein the semen is cooled to at least about 12° C.

58. The method of claim 42, wherein the fractionating step comprises incubating the ejaculate with a cell binding agent; permitting the sperm of a non selected sex type to preferentially bind to the cell binding agent; and separating the cell binding agent with preferentially bound sperm of the non selected sex type from non bound sperm, thereby providing a selected population of cells, the cell binding agent comprising a bead having a surface treatment comprising a material that preferentially binds to sperm of a non selected sex.

59. The method of claim 42, wherein the fractionating step comprises incubating a mixture of the ejaculate with a cell binding agent; permitting the sperm of a non selected sex type to preferentially bind to the cell binding agent; adding beads to the mixture, the beads being capable of binding with the cell binding agent; incubating the beads with the mixture to bind to the cell binding agent; and separating the beads and cell binding agent thereby preferentially removing sperm of the non selected sex type from the mixture, preferentially leaving sperm of a selected sex type.

60. A method for treating a specimen of semen containing sperm cells to increase the relative number of sperm cells of a preferred sex type in a treated specimen to increase the potential for conceiving an offspring of the preferred sex, the method comprising separating the semen into two components comprising a first component having a higher number of sperm of the preferred sex type than sperm of a non preferred sex type and a second component having a higher number of sperm of the non preferred sex type relative to sperm of the preferred sex type, wherein the separating step is performed in a window of time determined by locating a maximum in the curve obtained by plotting percent female cells determined by FISH against percent Koo positive cells, determining the time at which the maximum percent female cells occurs, and beginning the separation step no earlier than about one hour before the time of the maximum percent female cells.

61. The method of claim 60, wherein the separating step comprises contacting the sperm with a cell binding agent; permitting the sperm of the non preferred sex type to preferentially bind to the cell binding agent, and separating the cell binding agent with preferentially bound sperm of the non preferred sex type from non bound sperm, thereby providing said first component.

62. The method of claim 60, wherein the first component comprises a higher number of X-chromosome bearing sperm relative to Y-chromosome bearing sperm and the second component comprises a higher number of Y-chromosome bearing sperm relative to X-chromosome bearing sperm.

63. The method of claim 60, further comprising cooling the semen before performing the separating step.

64. The method of claim 60, further comprising collecting the semen and immediately cooling the semen.

65. The method of claim 64, wherein the semen is cooled to at least about 16° C.

66. The method of claim 64, wherein the semen is cooled to at least about 12° C.

67. A method for increasing the probability of producing an offspring of a mammalian species having a preferred sex, said method comprising: separating semen containing sperm cells of the mammalian species into two components comprising a first component having a higher number of sperm of the preferred sex type than sperm of a non preferred sex type and a second component having a higher number of sperm of the non preferred sex type relative to sperm of the preferred sex type, wherein the separating step is performed in a window of time determined by locating a maximum in the curve obtained by plotting percent female cells determined by FISH against percent Koo positive cells, determining the time at which the maximum percent female cells occurs, and beginning the separation step no earlier than about one hour before the time of the maximum percent female cells.

68. The method of claim 67, wherein the separating step comprises contacting the sperm with a cell binding agent; permitting the sperm of the non preferred sex type to preferentially bind to the cell binding agent, and separating the cell binding agent with preferentially bound sperm of the non preferred sex type from non bound sperm, thereby providing said first component.

69. The method of claim 67, wherein the first component comprises a higher number of X-chromosome bearing sperm relative to Y-chromosome bearing sperm and the second component comprises a higher number of Y-chromosome bearing sperm relative to X-chromosome bearing sperm.

70. The method of claim 67, further comprising cooling the semen before performing the separating step.

71. The method of claim 67, further comprising collecting the semen and immediately cooling the semen.

72. The method of claim 71, wherein the semen is cooled to about 12° C.

73. The method of claim 69, wherein the first component contains two-thirds viable X-chromosome bearing sperm and one-third viable Y-chromosome bearing sperm.

74. The method of claim 67, wherein twice as many female calves are born than male calves when the treated semen is used to fertilize a plurality of eggs.

75. A method for separating a selected population of cells from a sample of semen, the method comprising:
cooling the semen to a predetermined temperature;
waiting for a time determined by locating a maximum in the curve obtained by plotting percent female cells determined by FISH against percent Koo positive cells, determining the time at which the maximum percent female cells occurs, and beginning the following step no earlier than about one hour before the time of the maximum percent female cells;
contacting the sample with a cell binding agent to preferentially bind to the selected population of cells for a time sufficient for the cell binding agent to bind the selected cells; and
separating the selected cells and the cell binding agent to provide a treated sample containing non selected cells.

76. The method of claim 75, wherein the treated sample comprises a population of cells wherein a first number of cells exhibiting a preferred sex type is at least about 10% greater than a second number of cells exhibiting a non preferred sex type.

77. The method of claim 75, wherein the treated sample comprises a population of cells wherein a first number of cells exhibiting a preferred sex type is at least about 20% greater than a second number of cells exhibiting a non preferred sex type.

78. The method of claim 75, wherein the treated sample comprises a population of cells wherein a first number of cells exhibiting a preferred sex type is at least about 50% greater than a second number of cells exhibiting a non preferred sex type.

79. The method of claim 75, wherein the treated sample comprises a population of cells wherein a first number of cells exhibiting a preferred sex type is at least about 100% greater than a second number of cells exhibiting a non preferred sex type.

80. The method of claim 75, wherein the cell binding agent comprises a bead support having a diameter of 0.1 to 2 microns.

81. The method of claim 75, wherein the number of cells in the selected population of cells is greater than about 1×10⁵ cells/ml.

82. A method of insemination, the method comprising obtaining semen containing a population of spermatozoa according to the method of claim 75 and inseminating a mammal with the treated sample containing a population of spermatozoa.

83. The method of claim 82, wherein the mammal is selected from the group consisting of cattle, sheep, pigs, goats, horses, dogs and cats.

84. The method of claim 82, wherein the semen is cooled to at least about 16° C.

85. The method of claim 82, wherein the semen is cooled to at least about 12° C.

86. A method of increasing the percentage of mammalian offspring of a predetermined sex, the method comprising:
cooling a specimen of semen containing a population of cells to a predetermined temperature;
waiting for a time determined by locating a maximum in the curve obtained by plotting percent female cells determined by FISH against percent Koo positive cells, determining the time at which the maximum percent female cells occurs, and beginning the following step no earlier than about one hour before the time of the maximum percent female cells;
contacting the sample with a cell binding agent to preferentially bind to a selected population of cells for a time sufficient for the cell binding agent to bind the selected cells;
separating the selected cells and the cell binding agent to provide a treated sample containing non selected cells; and
administering at least a portion of the treated sample to the reproductive tract of a female mammal.

87. The method of claim 86, wherein the treated sample comprises a population of cells wherein a first number of cells exhibiting a preferred sex type is at least about 10% greater than a second number of cells exhibiting a non preferred sex type.

88. The method of claim 86, wherein the treated sample comprises a population of cells wherein a first number of cells exhibiting a preferred sex type is at least about 20% greater than a second number of cells exhibiting a non preferred sex type.

89. The method of claim 86, wherein the treated sample comprises a population of cells wherein a first number of cells exhibiting a preferred sex type is at least about 50% greater than a second number of cells exhibiting a non preferred sex type.

90. The method of claim 86, wherein the treated sample comprises a population of cells wherein a first number of cells exhibiting a preferred sex type is at least about 100% greater than a second number of cells exhibiting a non preferred sex type.

91. The method of claim 86, wherein the cell binding agent comprises a bead support having a diameter of 0.1 to 2 microns.

92. The method of claim 86, wherein the number of cells in the selected population of cells is greater than about 1×10⁵ cells/ml.

93. The method of claim 86, wherein the mammal is selected from the group consisting of cattle, sheep, pigs, goats, horses, dogs and cats.

94. The method of claim 93, wherein the semen is cooled to at least about 16° C.

95. The method of claim 93, wherein the semen is cooled to at least about 12° C.
